(19) United States
(12) Patent Application Publication
(10) Pub. No.: US 2011/0269810 A1
(43) Pub. Date: Nov. 3, 2011

(54) SELECTIVE INHIBITORS OF C-JUN N-TERMINAL KINASE

(75) Inventors: Yuan-Ping Pang, Rochester, MN (US); Anuradha Vummenthala, Iowa City, IA (US); Jewn Giew Park, Rochester, MN (US); Shao-Hua Wang, Rochester, MN (US); Zigang Dong, Rochester, MN (US); Ann M. Bode, Cannon Falls, MN (US); Yong Yeon Cho, Rochester, MN (US)

(73) Assignees: Regents of the University of Minnesota, Saint Paul, MN (US); Mayo Foundation for Medical Education and Research, Rochester, MN (US)

(21) Appl. No.: 13/079,254

(22) Filed: Apr. 4, 2011

Related U.S. Application Data
(63) Continuation-in-part of application No. PCT/US2009/058594, filed on Sep. 28, 2009.
(60) Provisional application No. 61/102,089, filed on Oct. 2, 2008.

(51) Int. Cl. A61K 31/403; A61P 19/02; A61P 3/10; A61P 25/28; C07D 209/56; C12N 9/99

(52) U.S. Cl. 514/410; 548/420; 435/184

(57) ABSTRACT

Compositions and methods for treating, preventing, or ameliorating one or more symptoms, disorders, or conditions associated with particular c-Jun N-terminal kinase(s) (JNKs) activity are provided. Compositions contain small molecules such as pyrazoloanthrones.

### CROSS-REFERENCE TO RELATED APPLICATIONS

This application is a continuation-in-part of PCT Patent Application Serial No. PCT/US2009/058594, filed on Sep. 28, 2009, which claims the benefit of U.S. Provisional Patent Application Ser. No. 61/102,089, filed on Oct. 2, 2008, both of which are incorporated herein by reference in their entirety.

### TECHNICAL FIELD

This disclosure relates to selective inhibitors of c-Jun N-terminal kinases (JNKs) and more particularly to small-molecules such as pyrazoloanthrones which are useful in the treatment of diseases related to JNK activity.

### BACKGROUND

c-Jun N-terminal kinases 1-3 (JNK1, JNK2, and JNK3) are serine/threonine protein kinases that phosphorylate c-Jun, a component of the activator protein-1 (AP-1) transcription factor complex. JNKs belong to the mitogen-activated protein kinase family of proteins and their signaling has been implicated in various diseases, including respiratory diseases, cancer, and neurological diseases. JNKs are encoded by three genes, Jnk1, Jnk2, and Jnk3, which are alternatively spliced to create at least 10 isoforms. Small molecule inhibitors of JNKs have been proposed for potential treatment of cancer, asthma, and Parkinson's disease. However, indiscriminately suppressing total JNKs activity may not be the appropriate strategy because each JNK may have a distinct function. Most studies focusing on cancer, neurological disease and respiratory disease have used JNK inhibitors that cannot differentiate between isoforms.

Developing selective inhibitors of JNK1, JNK2 or JNK3 can be challenging for at least two reasons. First, the three kinases share 90% sequence similarity and have the same amino acid sequence at the ATP-binding site. Known small-molecule inhibitors of JNKs such as SP600125 bind at the ATP-binding site and indiscriminately inhibit all three JNKs. Second, structural information for JNK1, JNK2, and JNK3 is lacking. The crystal structure of a truncated JNK1 (PDB code: 2P33) and JNK3 (PDB code: 2EXC) have been reported, whereas the X-ray structure of JNK2 has not been determined. The activation loop containing the dual phosphorylation site, Thr-Pro-Tyr, is missing in the crystal structure of JNK1. Using terascale supercomputing, three-dimensional (3D) models of JNK1, JNK2, and JNK3 with the activation loop have been developed. It was found that the activation-loop conformation of the JNKs can be markedly different between the three kinases, although the rest of the 3D structures are nearly identical.

Recent studies show that selective inhibitors of these kinases can provide treatment for devastating diseases. In particular, selective inhibitors of JNK1 can be used as therapeutics for treating type-2 diabetes (See: Liu et al. (2006) Bioorg. Med. Chem. Lett. 16: 2590-2594); selective inhibitors of both JNK1 and JNK2 can be used to treat rheumatoid arthritis (See: Alam et al. (2007) Bioorg. Med. Chem. Lett. 17: 3463-3467); selective inhibitors of JNK3 are potential drugs for treating neural degeneration associated with conditions such as Parkinson's disease, multiple sclerosis, and Alzheimer's disease (See: Graczyk et al. (2005) Bioorg. Med. Chem. Lett. 15: 4666-4670 & Swahn et al. (2005) Bioorg. Med. Chem. Lett. 15: 5095-5099).

### SUMMARY

Accordingly, the disclosure provided herein relates to materials and methods for treating, preventing, or ameliorating one or more symptoms, disorders, or conditions associated with particular c-Jun N-terminal kinases (JNKs) and their activities. The present invention provides selective inhibitors of JNK1, JNK3 and JNKs1&2 as compared to JNK3. In one aspect of the disclosure, a composition of matter includes a compound of Formula I:

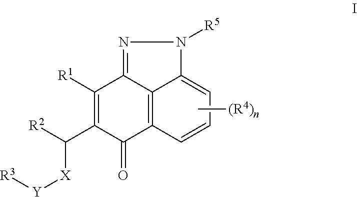

or pharmaceutically acceptable salts thereof, wherein constituent members are defined herein.

The invention further provides a method of modulating (such as, inhibiting) an activity of JNK1 including, contacting JNK1 with a compound of Formula I, or a pharmaceutically acceptable salt thereof.

The invention further provides a method of modulating (such as, inhibiting) an activity of JNK3 including, contacting JNK3 with a compound of Formula I, or a pharmaceutically acceptable salt thereof.

The invention further provides a method of modulating (such as, inhibiting) an activity of JNK1 and JNK2 including, contacting JNK1 and JNK2 with a compound of Formula I, or a pharmaceutically acceptable salt thereof.

The invention further provides a method for treating, preventing, or ameliorating one or more symptoms associated with type-2 diabetes including, administering to a subject in need thereof a therapeutically effective amount of a compound of Formula I, or a pharmaceutically acceptable salt thereof.

The invention further provides a method for treating, preventing, or ameliorating one or more symptoms associated with insulin resistance including, administering to a subject in need thereof a therapeutically effective amount of a compound of Formula I, or a pharmaceutically acceptable salt thereof.

The invention further provides a method for treating, preventing, or ameliorating one or more symptoms associated with neural degeneration (such as, Parkinson's disease, multiple sclerosis, or Alzheimer's disease) including, administering to a subject in need thereof a therapeutically effective amount of a compound of Formula I, or a pharmaceutically acceptable salt thereof.

The invention further provides a method for treating, preventing, or ameliorating one or more symptoms associated with rheumatoid arthritis including, administering to a subject in need thereof a therapeutically effective amount of a compound of Formula I, or a pharmaceutically acceptable salt thereof.

The invention further provides a compound of Formula I, or a pharmaceutically acceptable salt thereof, for use in therapy.

The invention further provides use of a compound of Formula I, or a pharmaceutically acceptable salt thereof, for the production of a medicament for use in therapy.

Unless otherwise defined, all technical and scientific terms used herein have the same meaning as commonly understood by one of ordinary skill in the art to which this invention belongs. Although methods and materials similar or equivalent to those described herein can be used in practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1. Inhibitory effect of 14a on JNK1, JNK2, and JNK3 activity.

FIG. 2. Inhibitory effect of 14b-d on JNK1, JNK2, and JNK3 activity.

FIG. 3. Inhibitory effect of 9a-b on JNK1, JNK2, and INK3 activity.

FIG. 4. Inhibitory effect of 11 on JNK1, JNK2, and JNK3 activity.

### DETAILED DESCRIPTION

The inventions disclosed herein pertain to identification, syntheses, and verification of (1) a class of small molecules that showed selective inhibition to JNK1 but not to JNK2 and JNK3; (2) a class of small molecules that showed selective inhibition to both JNK1 and JNK2 but not to JNK3; (3) a class of small molecules that showed selective inhibition to JNK3 but not to JNK1 and JNK2. Selective inhibition of JNK1, JNK2, or JNK3 is expected to result in differential effects on various parameters to be assessed in cell culture and animal models of asthma, cancer, and neurological disease, respectively. These molecules can be used as therapeutics for treating type-2 diabetes, rheumatoid arthritis, Parkinson's disease, multiple sclerosis, and Alzheimer's disease and also as reagents for investigating the roles of JNKs in various disease states.

### A. Definitions

As used herein, pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, nitrates, borates, methanesulfonates, benzenesulfonates, toluenesulfonates, salts of mineral acids, such as but not limited to hydrochlorides, hydrobromides, hydroiodides and sulfates; and salts of organic acids, such as but not limited to acetates, trifluoroacetates, maleates, oxalates, lactates, malates, tartrates, citrates, benzoates, salicylates, ascorbates, succinates, butyrates, valerates and fumarates.

As used herein, treatment means any manner in which one or more of the symptoms related to a JNK1, JNK2, or JNK3 activity, e.g., type-2 diabetes or symptoms associated with insulin resistance; neural degeneration; or rheumatoid arthritis, are ameliorated or otherwise beneficially altered. Treatment also encompasses any pharmaceutical use of the compositions herein, such as uses for treating diseases, disorders, or ailments in which JNK1, JNK2, or JNK3 is implicated.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, IC₅₀ refers to an amount, concentration or dosage of a particular test compound that achieves a 50% inhibition of a maximal response in an assay that measures such response.

As used herein, the term Kᵢ represents the dissociation constant of an enzyme/inhibitor complex. It is theoretically independent of the substrate against which the inhibitor is tested. Kᵢ can be calculated from an IC₅₀ using the equation:
Kᵢ=IC₅₀*Kₘ/(S+Kₘ), where S is the concentration of substrate, and Kₘ is the substrate concentration (in the absence of inhibitor) at which the velocity of the reaction is half-maximal. The Kᵢ of an inhibitor for inhibition of a particular substrate (fixed Kₘ) is constant.

As used herein, EC₅₀ refers to a drug concentration that produces 50% of inhibition, and CC₅₀ refers to a drug concentration that produces 50% of toxicity.

It is to be understood that the compounds provided herein may contain chiral centers. Such chiral centers may be of either the (R) or (S) configuration, or may be a mixture thereof In certain cases, particular R and S configurations may be preferred. Thus, the compounds provided herein may be enantiomerically pure, or be stereoisomeric or diastereomeric mixtures. In the case of amino acid residues, such residues may be of either the L- or D-form. The configuration for naturally occurring amino acid residues is generally L. When not specified the residue is the L form. As used herein, the term “amino acid” refers to a-amino acids which are racemic, or of either the D- or L-configuration. The designation “d” preceding an amino acid designation (e.g., dAla, dSer, dVal, etc.) refers to the D-isomer of the amino acid. The designation “dl” preceding an amino acid designation refers to a mixture of the L- and D-isomers of the amino acid. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC), and mass spectrometry (MS), used by those of skill in the art to assess such purity, or sufficiently pure such that further purification would not detectably alter the physical and chemical properties, such as melting point, enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, “alkyl,” “alkenyl” and “alkynyl” refer to carbon chains that may be straight or branched. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, vinyl, allyl (propenyl), homoallyl, butadienyl, isoprenyl, ethynyl, and propargyl (propynyl).

As used herein, “cycloalkyl” refers to a saturated or unsaturated, mono- or multi-cyclic ring system, in certain embodiments of 3 to 10 carbon atoms, in other embodiments of 3 to 6 carbon atoms. The ring systems of the cycloalkyl groups may be composed of one ring or two or more rings which may be joined together in a fused, bridged or spiro-connected fashion. Examples include, but are not limited to, cyclopropyl, cyclobutyl, cyclopentyl, and cyclohexyl.

As used herein, “aryl” refers to aromatic monocyclic or multicyclic groups containing from 6 to 19 carbon atoms. Aryl groups include, but are not limited to groups such as unsubstituted or substituted fluorenyl, unsubstituted or substituted phenyl, and unsubstituted or substituted naphthyl.

As used herein, “heteroaryl” refers to a monocyclic or multicyclic aromatic ring system, in certain embodiments, of about 5 to about 15 members, where one or more, in one embodiment 1 to 4, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

As used herein, “heterocycloalkyl” refers to a monocyclic or multicyclic, saturated or unsaturated ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur.

As used herein, “alkylene,” “alkenylene,” “alkynylene,” “cycloalkylene,” “arylene,” “heteroarylene,” and “heterocycloalkylene” refer to divalent linking “alkyl,” “alkenyl,” “alkynyl,” “cycloalkyl,” “aryl,” “heteroaryl,” and “heterocycloalkyl” groups.

As used herein, “halo”, “halogen” or “halide” refers to F, Cl, Br or I.

As used herein, “haloalkyl” refers to an alkyl group in which one or more of the hydrogen atoms are replaced by halogen.

As used herein, “aminoalkyl” refers to —RNH₂, in which R is alkyl and the linkage is through a carbon atom.

As used herein, “alkoxy” refers to RO— in which R is alkyl.

As used herein, “alkylamino” refers to RNH—, in which R is alkyl and the linkage is through a nitrogen atom.

As used herein, “dialkylamino” refers to R(R′)N—, in which R and R′ are the same or different alkyl and the linkage is through a nitrogen atom.

Where the number of any given substituent is not specified (e.g., haloalkyl), there may be one or more substituents present. For example, “haloalkyl” may include one or more of the same or different halogens.

As used herein, the abbreviations for any protective groups, amino acids and other compounds, are, unless indicated otherwise, in accord with their common usage, recognized abbreviations, or the IUPAC-IUB Commission on Biochemical Nomenclature (See, (1972) Biochem. 11:942-944).

### B. Compositions of Matter

Provided herein are methods and compositions for treating, preventing, or ameliorating one or more symptoms, disorders, or conditions associated with particular JNK activity, e.g., JNK1, JNK2, or JNK3.

In one aspect of the disclosure is provided a compound of Formula I:

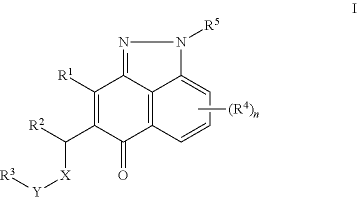

wherein:

R¹ and R⁴ are independently selected from H, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}S(O)₂R^{b1}, and S(O)₂NR^{c1}R^{d1}; or

R¹ and R⁴ are independently selected from C₁₋₁₀ alkyl, C₂₋₆ alkenyl, C₂₋₆ alkynyl, C₆₋₁₂ aryl, C₅₋₁₂ heteroaryl, C₃₋₁₂ cycloalkyl, C₃₋₁₀ heterocycloalkyl, heterocycloalkylalkyl, arylalkyl, and heteroarylalkyl, each optionally substituted with 1, 2, 3, 4, or 5 substituents selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1};

R² is selected from H, C(O)R^{b1}, C(O)NR^{c1}R^{d1}, and C(O)OR^{a1}; or

R² is selected from C₁₋₁₀ alkyl, C₂₋₆ alkenyl, C₂₋₆ alkynyl, C₆₋₁₂ aryl, C₅₋₁₂ heteroaryl, C₃₋₁₂ cycloalkyl, C₃₋₁₀ heterocycloalkyl, heterocycloalkylalkyl, arylalkyl, and heteroarylalkyl, each optionally substituted with 1, 2, 3, 4, or 5 substituents selected from halo, CN, NO₂, OR^{a1}, SR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1};

or,

R¹ and R² together with the three C atoms between them may form a 5 or 6 membered cycloalkyl, aryl, or heteroaryl ring each optionally substituted with 1, 2, or 3 substituents independently selected from C₁₋₆ alkyl, C₂₋₆ alkenyl, C₂₋₆ alkynyl, C₁₋₆ alkoxy, C₁₋₆ haloalkyl, halo, OH, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1};

R³ is selected from OR^{a2}, SR^{a2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)₂R^{b2}, C(O)C₁₋₆ alkyl, C(O)C₆₋₁₂ aryl, C(O)NR^{c2}R^{d2}, C(O)OR^{a2}, OC(O)R^{b2}, OC(O)NR^{c2}R^{d2}, S(O)R^{b2}, S(O)NR^{c2}R^{d2}, S(O)₂R^{b2}, and S(O)₂NR^{c2}R^{d2};

X═O, S, C(O), or NR⁶;

Y is a divalent moiety selected from C₃₋₁₂ alkylene, C₂₋₁₀ alkenylene, C₂₋₈ alkynylene, C₃₋₁₀ cycloalkylene, C₃₋₁₀ heterocycloalkylene, C₆₋₁₀ arylene, and C₅₋₁₀ heteroarylene, each optionally substituted by 1, 2 or 3 substituents independently selected from C₁₋₄ alkyl, C₁₋₄ hydroxyalkyl, C₁₋₄ cyanoalkyl, C₁₋₄ haloalkyl, C₁₋₄ alkoxy-C₁₋₄ alkyl, C₂₋₄ alkenyl, C₂₋₄ alkynyl, C₃₋₁₀ cycloalkyl, C₃₋₁₀ heterocycloalkyl, C₆₋₁₀ aryl, C₅₋₁₀ heteroaryl, halo, CN, NO₂, SCN, OH, C₁₋₄alkoxy, C₁₋₄ haloalkoxy, amino, C₁₋₄ alkylamino, and C₂₋₈ dialkylamino;

R⁵ and R⁶ are independently selected from H, C₁₋₆ alkyl, C₁₋₄ alkoxy-C₁₋₄ alkyl, C(O)C₁₋₆ alkyl, aryl, heteroaryl, C₇₋₁₈ arylalkyl, and C(O)C₆₋₁₂ aryl;

R^{a1} and R^{a2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

R^{b1} and R^{b2} are independently selected from H, C₁₋₆ alkyl, C₁₋₆ haloalkyl, C₂₋₆ alkenyl, C₂₋₆ alkynyl, aryl, cycloalkyl, heteroaryl, and heterocycloalkyl, wherein said C₁₋₆ alkyl, C₂₋₆ alkenyl, C₂₋₆ alkynyl, aryl, cycloalkyl, heteroaryl, or heterocycloalkyl is optionally substituted with 1, 2, or 3 substituents independently selected from OH, C₁₋₆ alkoxy, CN, amino, alkylamino, dialkylamino, halo, C₁₋₆ alkyl, C₁₋₆ haloalkyl, aryl, arylalkyl, heteroaryl, heteroarylalkyl, cycloalkyl, and heterocycloalkyl;

R^{c1}, R^{c2}, R^{d1}, and R^{d2} are independently selected from H, C₁₋₆ alkyl, C₂₋₆ alkenyl, C₂₋₆ alkynyl, aryl, cycloalkyl, heteroaryl, and heterocycloalkyl, wherein said C₁₋₆ alkyl, C₂₋₆ alkenyl, C₂₋₆ alkynyl, aryl, cycloalkyl, heteroaryl, or heterocycloalkyl is optionally substituted with 1, 2, or 3 substituents independently selected from OH, C₁₋₆ alkoxy, CN, amino, alkylamino, dialkylamino, halo, C₁₋₆ alkyl, C₁₋₆ haloalkyl, aryl, arylalkyl, heteroaryl, heteroarylalkyl, cycloalkyl, and heterocycloalkyl; or,

R^{c1} and R^{d1}, or R^{c2} and R^{d2}, together with the N atom to which they are attached, may optionally form a 4-, 5-, 6-, or 7-membered heterocycloalkyl group or heteroaryl group, each optionally substituted with 1, 2, or 3 substituents independently selected from OH, C₁₋₆ alkoxy, CN, amino, alkylamino, dialkylamino, halo, C₁₋₆ alkyl, C₁₋₆ haloalkyl, aryl, arylalkyl, heteroaryl, heteroarylalkyl, cycloalkyl, and heterocycloalkyl; and

n is 1, 2, or 3; or a pharmaceutically acceptable salt thereof.

In some embodiments, R¹ and R⁴ are independently selected from H, halo, CN, NO₂, OR^{a1}, and SR^{a1}.

In some embodiments, R¹ and R⁴ are independently selected from H, C(O)R^{b1}, C(O)NR^{c1}R^{d1}, C(O)OR^{a1}, OC(O)R^{b1}, and OC(O)NR^{c1}R^{d1}.

In some embodiments, R¹ and R⁴ are independently selected from H, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}.

In some embodiments, R¹ and R⁴ are independently selected from C₁₋₁₀ alkyl, C₂₋₆ alkenyl, C₂₋₆ alkynyl, C₆₋₁₂ aryl, C₃₋₁₂ cycloalkyl, and arylalkyl, each optionally substituted with 1, 2, 3, 4, or 5 substituents selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}.

In some embodiments, R¹ and R⁴ are independently selected from C₅₋₁₂ heteroaryl, C₃₋₁₀ heterocycloalkyl, heterocycloalkylalkyl, and heteroarylalkyl, each optionally substituted with 1, 2, 3, 4, or 5 substituents selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}.

In some embodiments, R² is selected from H, C(O)R^{b1}, C(O)NR^{c1}R^{d1}, and C(O)OR^{a1}.

In some embodiments, R² is selected from C₁₋₁₀ alkyl, C₂₋₆ alkenyl, C₂₋₆ alkynyl, C₆₋₁₂ aryl, C₃₋₁₂ cycloalkyl, and arylalkyl, each optionally substituted with 1, 2, 3, 4, or 5 substituents selected from halo, CN, NO₂, OR^{a1}, SR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}.

In some embodiments, R² is selected from C₅₋₁₂ heteroaryl, C₃₋₁₀ heterocycloalkyl, heterocycloalkylalkyl, and heteroarylalkyl, each optionally substituted with 1, 2, 3, 4, or 5 substituents selected from halo, CN, NO₂, OR^{a1}, SR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}.

In some embodiments, R¹ and R² together with the three C atoms between them may form a 5, 6, or 7 membered cycloalkyl ring optionally substituted with 1, 2, or 3 substituents independently selected from C₁₋₆ alkyl, C₂₋₆ alkenyl, C₂₋₆ alkynyl, C₁₋₆ alkoxy, C₁₋₆ haloalkyl, halo, OH, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}.

In some embodiments, R¹ and R² together with the three C atoms between them may form a 6 membered aryl ring optionally substituted with 1, 2, or 3 substituents independently selected from C₁₋₆ alkyl, C₂₋₆ alkenyl, C₂₋₆ alkynyl, C₁₋₆ alkoxy, C₁₋₆ haloalkyl, halo, OH, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}.

In some embodiments, ¹ and R² together with the three C atoms between them may form a 5 or 6 membered heteroaryl ring optionally substituted with 1, 2, or 3 substituents independently selected from C₁₋₆ alkyl, C₂₋₆ alkenyl, C₂₋₆ alkynyl, C₁₋₆ alkoxy, C₁₋₆ haloalkyl, halo, OH, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}.

In some embodiments, R¹ and R² together with the three C atoms between them may form a 6-membered aryl ring optionally substituted with 1, 2, or 3 substituents independently selected from C₁₋₆ alkyl, C₂₋₆ alkenyl, C₂₋₆ alkynyl, C₁.₆ alkoxy, C₁₋₆ haloalkyl, halo, OH, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}.

In some embodiments, R³ is selected from NHCHO, OR^{a2}, SR^{a2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)₂R^{b2}, C(O)C₁₋₆ alkyl, C(O)C₆₋₁₂ aryl, C(O)NR^{c2}R^{d2}, C(O)OR^{a2}, OC(O)R^{b2}, OC(O)NR^{c2}R^{d2}, S(O)R^{b2}, S(O)NR^{c2}R^{d2}, S(O)₂R^{b2}, and S(O)₂NR^{c2}R^{d2}.

In some embodiments, R³ is selected from OR^{a2}, SR^{a2}, NR^{c2}R^{d2}, NR^{c2}C(O)R^{b2}, NR^{c2}C(O)NR^{c2}R^{d2}, NR^{c2}C(O)OR^{a2}, and NR^{c2}S(O)₂R^{b2}.

In some embodiments, R³ is selected from OR^{a2}, NR^{c2}R^{d2}, NR^{c2}C(O)R^{b2}, NR^{c2}C(O)OR^{a2}, C(O)C₁₋₆ alkyl, C(O)C₆₋₁₂ aryl, C(O)NR^{c2}R^{d2}, C(O)OR^{a2}, OC(O)R^{b2}, OC(O)NR^{c2}R^{d2}, S(O)R^{b2}, S(O)NR^{c2}R^{d2}, S(O)₂R^{b2}, and S(O)₂NR^{c2}R^{d2}.

In some embodiments, R³ is selected from OR^{a2}, NR^{c2}R^{d2}, NR^{c2}C(O)R^{b2}, and NR^{c2}C(O)OR^{a2}.

In some embodiments, R³ is NH—CHO.

In some embodiments, X═O, S, C(O), or NR⁶.

In some embodiments, X═O.

In some embodiments, X═S.

In some embodiments, X═C(O).

In some embodiments, X═NR⁶.

In some embodiments, Y is a divalent moiety selected from C₃₋₁₂ alkylene, C₂₋₁₀ alkenylene, C₂₋₈ alkynylene, C₃₋₁₀ cycloalkylene, and C₆₋₁₀ arylene, each optionally substituted by 1, 2 or 3 substituents independently selected from Cᵢ₋₄ alkyl, C₁₋₄ hydroxyalkyl, C₁₋₄ cyanoalkyl, C₁₋₄ haloalkyl, C₁₋₄ alkoxy-C₁₋₄ alkyl, C₂₋₄ alkenyl, C₂₋₄ alkynyl, C₃₋₁₀ cycloalkyl, C₃₋₁₀ heterocycloalkyl, C₆₋₁₀ aryl, C₅₋₁₀ heteroaryl, halo, CN, NO₂, SCN, OH, C₁₋₄alkoxy, C₁₋₄ haloalkoxy, amino, C₁₋₄ alkylamino, and C₂₋₈ dialkylamino.

In some embodiments, Y is a divalent moiety selected from C₃₋₁₂ alkylene, C₃₋₁₀ heterocycloalkylene, and C₅₋₁₀ heteroarylene, each optionally substituted by 1, 2 or 3 substituents independently selected from C₁₋₄ alkyl, C₁₋₄ hydroxyalkyl, C₁₋₄ cyanoalkyl, C₁₋₄ haloalkyl, C₁₋₄ alkoxy-C₁₋₄ alkyl, C₂₋₄ alkenyl, C₂₋₄ alkynyl, C₃₋₁₀ cycloalkyl, C₃₋₁₀ heterocycloalkyl, C₆₋₁₀ aryl, C₅₋₁₀ heteroaryl, halo, CN, NO₂, SCN, OH, C₁₋₄alkoxy, C₁₋₄ haloalkoxy, amino, C₁₋₄ alkylamino, and C₂₋₈ dialkylamino.

In some embodiments, Y is a divalent moiety selected from C₃₋₁₂ alkylene optionally substituted by 1, 2 or 3 substituents independently selected from C₁₋₄ alkyl, C₁₋₄ hydroxyalkyl, C₁₋₄ cyanoalkyl, C₁₋₄ haloalkyl, C₁₋₄ alkoxy-C₁₋₄ alkyl, C₂₋₄ alkenyl, C₂₋₄ alkynyl, C₃₋₁₀ cycloalkyl, C₃₋₁₀ heterocycloalkyl, C₆₋₁₀ aryl, C₅₋₁₀ heteroaryl, halo, CN, NO₂, SCN, OH, C₁₋₄alkoxy, C₁₋₄ haloalkoxy, amino, C₁₋₄ alkylamino, and C₂₋₈ dialkylamino.

In some embodiments, R⁵ and R⁶ are independently selected from H, C₁₋₆ alkyl, C₁₋₄ alkoxy-C₁₋₄ alkyl, C(O)C₁₋₆ alkyl, phenyl, aryl, heteroaryl, C₇₋₁₈ arylalkyl, and C(O)C₆₋₁₂ aryl.

In some embodiments, R⁵ and R⁶ are independently selected from I-1, C₁₋₄ alkoxy-C₁₋₄ alkyl, C(O)C₁₋₆ alkyl, C₇₋₁₈ arylalkyl, and C(O)C₆₋₁₂ aryl.

In some embodiments, R⁵ and R⁶ are independently selected from H, C₁₋₆ alkyl, aryl, and heteroaryl.

In some embodiments, R⁵ and R⁶ are independently H or C₇₋₁₈ arylalkyl.

In some embodiments, R⁵ is aryl.

In some embodiments, R⁵ is phenyl.

In some embodiments, n is 1.

In some embodiments, n is 2.

In some embodiments, n is 3.

In some embodiments, the compounds of the invention have the Formula II:

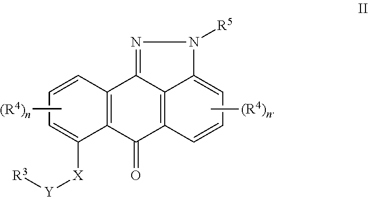

In some embodiments, the compounds of the invention have the Formula III:

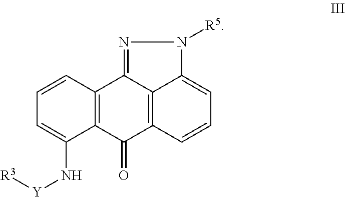

In some embodiments, the compounds of the invention have the Formula VI:

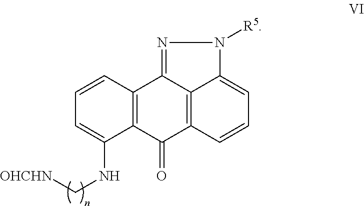

In another aspect of the disclosure is provided a compound of Formula II:

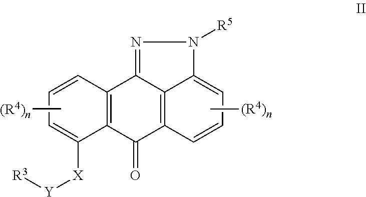

wherein:

R³ is selected from OR^{a2}, SR^{a2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)₂R^{b2}, C(O)C₁₋₆ alkyl, C(O)C₆₋₁₂ aryl, C(O)NR^{c2}R^{d2}, C(O)OR^{a2}, OC(O)R^{b2}, OC(O)NR^{c2}R^{d2}, S(O)R^{b2}, S(O)NR^{c2}R^{d2}, S(O)₂R^{b2}, and S(O)₂NR^{c2}R^{d2};

R⁴ is selected from H, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}; or

R⁴ is selected from C₁₋₁₀ alkyl, C₂₋₆ alkenyl, C₂₋₆ alkynyl, C₆₋₁₂ aryl, C₅₋₁₂ heteroaryl, C₃₋₁₂ cycloalkyl, C₃₋₁₀ heterocycloalkyl, heterocycloalkylalkyl, arylalkyl, and heteroarylalkyl, each optionally substituted with 1, 2, 3, 4, or 5 substituents selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1};

X═O, S, C(O), or NR⁶;

Y is a divalent moiety selected from C₃₋₁₂ alkylene, C₂₋₁₀ alkenylene, C₂₋₈ alkynylene, C₃₋₁₀ cycloalkylene, C₃₋₁₀ heterocycloalkylene, C₆₋₁₀ arylene, and C₅₋₁₀ heteroarylene, each optionally substituted by 1, 2 or 3 substituents independently selected from C₁₋₄ alkyl, C₁₋₄ hydroxyalkyl, C₁₋₄ cyanoalkyl, C₁₋₄ haloalkyl, C₁₋₄ alkoxy-C₁₋₄ alkyl, C₂₋₄ alkenyl, C₂₋₄ alkynyl, C₃₋₁₀ cycloalkyl, C₃₋₁₀ heterocycloalkyl, C₆₋₁₀ aryl, C₅₋₁₀ heteroaryl, halo, CN, NO₂, SCN, OH, C₁₋₄alkoxy, C₁₋₄ haloalkoxy, amino, C₁₋₄ alkylamino, and C₂₋₈ dialkylamino;

R⁵ and R⁶ are independently selected from H, C₁₋₆ alkyl, C₁₋₄ alkoxy-C₁₋₄ alkyl, C(O)C₁₋₆ alkyl, aryl, heteroaryl, C₇₋₁₈ arylalkyl, and C(O)C₆₋₁₂ aryl;

R^{a1} and R^{a2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

R^{b1} and R^{b2} are independently selected from H, C₁₋₆ alkyl, C₁₋₆ haloalkyl, C₂₋₆ alkenyl, C₂₋₆ alkynyl, aryl, cycloalkyl, heteroaryl, and heterocycloalkyl, wherein said C₁₋₆ alkyl, C₂₋₆ alkenyl, C₂₋₆ alkynyl, aryl, cycloalkyl, heteroaryl, or heterocycloalkyl is optionally substituted with 1, 2, or 3 substituents independently selected from OH, C₁₋₆ alkoxy, CN, amino, alkylamino, dialkylamino, halo, C₁₋₆ alkyl, C₁₋₆ haloalkyl, aryl, arylalkyl, heteroaryl, heteroarylalkyl, cycloalkyl, and heterocycloalkyl;

R^{c1}, R^{c2}, R^{d1}, and R^{d2} are independently selected from H, C₁₋₆ alkyl, C₂₋₆ alkenyl, C₂₋₆ alkynyl, aryl, cycloalkyl, heteroaryl, and heterocycloalkyl, wherein said C₁₋₆ alkyl, C₂₋₆ alkenyl, C₂₋₆ alkynyl, aryl, cycloalkyl, heteroaryl, or heterocycloalkyl is optionally substituted with 1, 2, or 3 substituents independently selected from OH, C₁₋₆ alkoxy, CN, amino, alkylamino, dialkylamino, halo, C₁₋₆ alkyl, C₁₋₆ haloalkyl, aryl, arylalkyl, heteroaryl, heteroarylalkyl, cycloalkyl, and heterocycloalkyl; or,

R^{c1} and R^{d1}, or R^{c2} and R^{d2}, together with the N atom to which they are attached, may optionally form a 4-, 5-, 6-, or 7-membered heterocycloalkyl group or heteroaryl group, each optionally substituted with 1, 2, or 3 substituents independently selected from OH, C₁₋₆ alkoxy, CN, amino, alkylamino, dialkylamino, halo, C₁₋₆ alkyl, C₁₋₆ haloalkyl, aryl, arylalkyl, heteroaryl, heteroarylalkyl, cycloalkyl, and heterocycloalkyl; and

n is 1, 2, or 3; or a pharmaceutically acceptable salt thereof.

In some embodiments, R⁴ is selected from H, halo, CN, NO₂, OR^{a1}, and SR^{a1}.

In some embodiments, X═NR⁶.

In some embodiments, R³ is selected from OR^{a2}, NR^{c2}R^{d2}, NR^{c2}C(O)R^{b2}, and NR^{c2}C(O)OR^{a2}.

In some embodiments, Y is C₃₋₁₂ alkylene optionally substituted by 1, 2 or 3 substituents independently selected from C₁₋₄ alkyl, C₁₋₄ hydroxyalkyl, C₁₋₄ cyanoalkyl, C₁₋₄ haloalkyl, C₁₋₄ alkoxy-C₁₋₄ alkyl, C₂₋₄ alkenyl, C₂₋₄ alkynyl, C₃₋₁₀ cycloalkyl, C₃₋₁₀ heterocycloalkyl, C₆₋₁₀ aryl, C₅₋₁₀ heteroaryl, halo, CN, NO₂, SCN, OH, C₁₋₄alkoxy, C₁₋₄ haloalkoxy, amino, C₁₋₄ alkylamino, and C₂₋₈ dialkylamino.

In some embodiments, R⁵ and R⁶ are independently H or C₇₋₁₈ arylalkyl.

In some embodiments, n is 1.

In some embodiments, n is 2.

In some embodiments, n is 3.

### C. Preparation of the Compounds

The compounds of the present invention can be prepared in a variety of ways known to one skilled in the art of synthetic organic chemistry or by the methods described herein. In addition, certain variations to the procedures with respect to reaction conditions such as stoichiometry, solvents, reagents, catalysts, and temperatures; work-up; and purification conditions described below will be recognized by one skilled in the art. Preparation of the compounds can involve a temporary protection and deprotection of reactive chemical groups. The chemistry of protecting groups can be found in Wuts and Greene, Greene's Protective Groups in Organic Synthesis, 4^{th} Ed., Wiley & Sons: New York, 2006.

The compounds of the invention can be prepared, for example, by the Schemes 1-4 shown below.

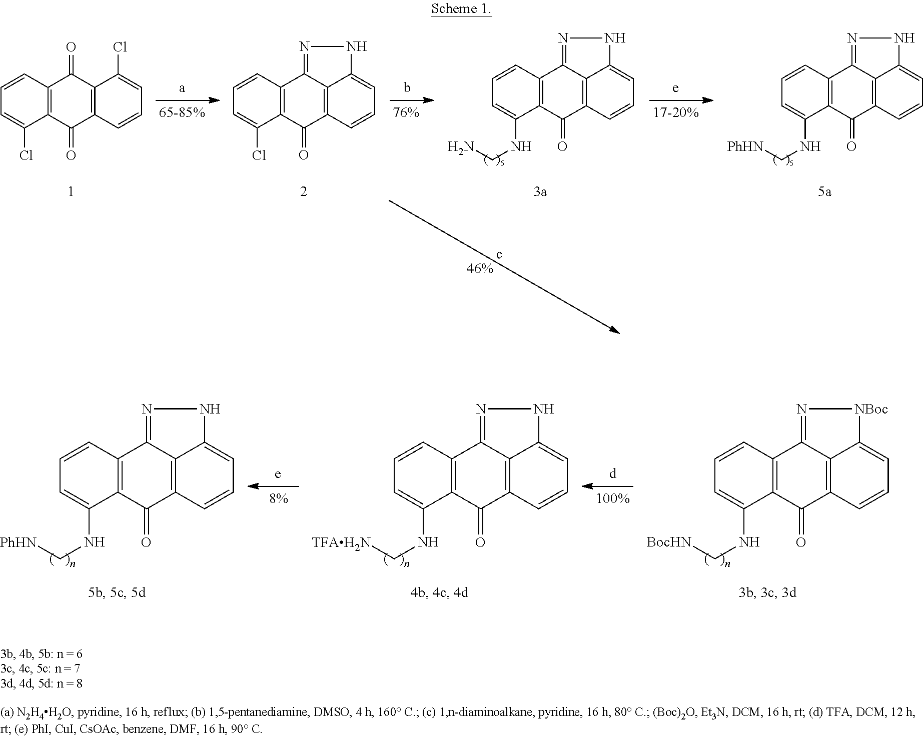

The 1,5-dichloroanthraquinone 1 can be treated with hydrazine to form a pyrazoloanthrone 2 which can undergo aromatic nucleophilic substitution with a diamine to provide a 7-aminopyrazoloanthrone 3. In the case of a 1,5-pentanediamine adduct 3a, no protection was required and the adduct 3a was converted directly to the phenyl adduct 5a. In the case of 1,6-hexanediamine or higher homologs, the amine adducts were initially isolated as di-Boc-protected amines 3b-d followed by deprotection under acidic conditions to form TFA salts 4b-d. The coupling of the amine salts 4b-d can be carried out in the presence of a base and a catalyst to provide the desired 5b-d.

The products obtained by the synthesis outlined in Scheme 1 were originally determined, based on ¹H NMR spectral analysis, to possess the structures represented as 5a-d in which the phenyl group is attached to the terminal alkylene-amino group. However, further studies—including high resolution mass spectroscopic analysis (HRMS)—established that the compounds represented by structures 5a-d were, in fact, compounds depicted in structures 14a-d in which the phenyl group is attached to the nitrogen atom on the pyrazole ring (See, Scheme 4). This was also further confirmed by an alternate independent synthesis of compound with structure 5b (See, Scheme 5) and comparing its spectral (NMR and mass) information with compound 14b.

In an alternative procedure to obtain compounds 9a-b, Scheme 2 was utilized.

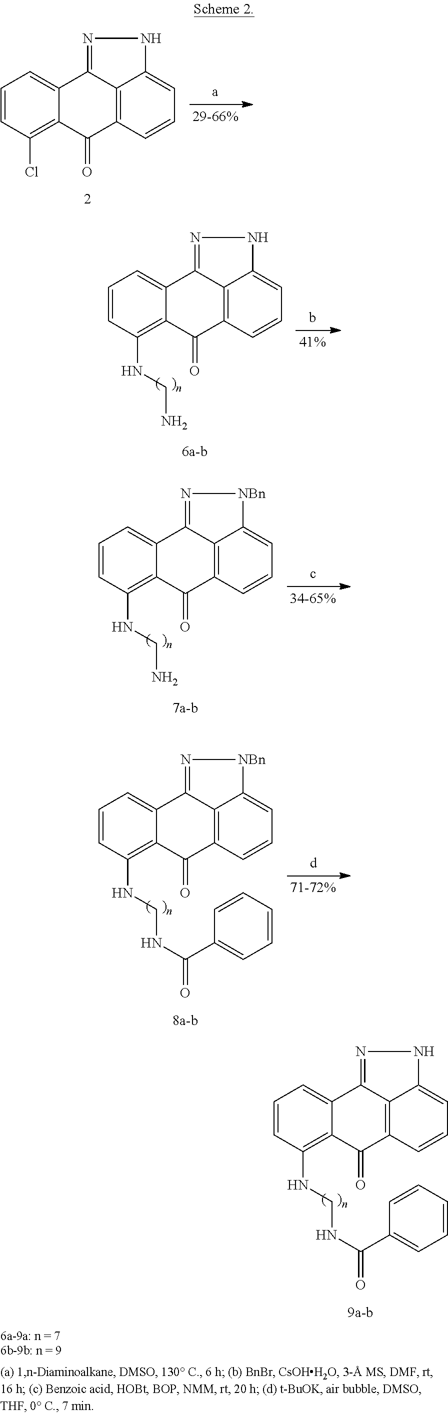

The NH of the pyrazole ring in this procedure can be protected as a benzyl group shown in 7a-b before benzoylation with benzoic acids using standard protocols. A longer alkylene spacer can be utilized in these examples.

The compound 11 was prepared according to Scheme 3 where compound 2 was treated with an aminopentanol followed by a treatment of the free hydroxyl group with p-methylphenol (p-cresol) to provide the compound 11.

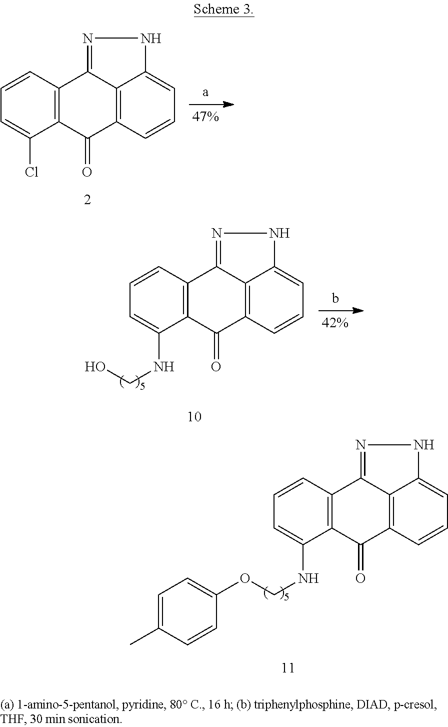

The compounds 14a-d can also be prepared by an alternate route, according to the Scheme 4. The 1,5-dichloroanthraquinone 1 can be treated with hydrazine to form a pyrazoloanthrone 2 which can undergo aromatic nucleophilic substitution with iodobenzene to provide 7-chloro-2-phenylanthra[1,9-cd]pyrazol-6-one 12. Using different alkylenediamines, compound 12 can be converted to amine adducts which can subsequently be obtained as TFA salts 13a-d after HPLC purification with TFA. Formylation of the amine salts 13a-d can be achieved in the presence of sulfuric acid and DMF to provide 14a-d.

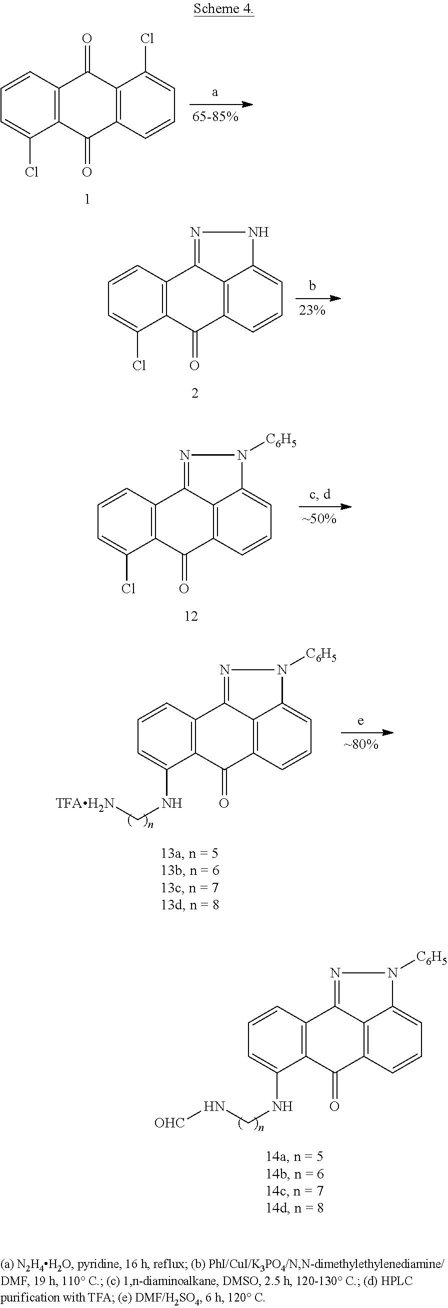

Compound 5b can be prepared by an alternative route outlined in Scheme 5 to obtain a sample for NMR an mass spectral analysis. Treatment of aniline with protected aminoalkylene bromide can provide the compound 15 which upon deprotection can provide compound 16 which can be reacted with 2 under refluxing conditions to provide an authentic sample of 5b.

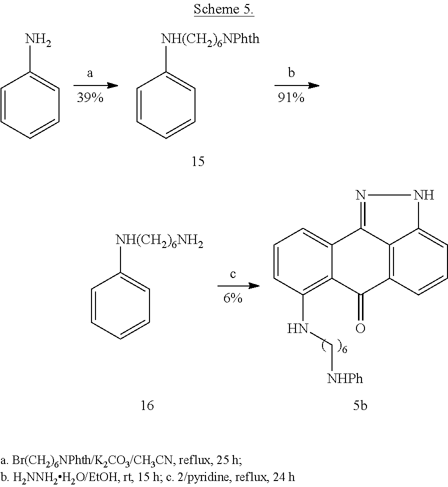

The compounds of the invention were characterized using standard techniques such as melting point, nuclear magnetic resonance (NMR) and MS including HRMS. In general, ¹H NMR spectra were recorded on a Varian Mercury 400 spectrometer. Chemical shifts are reported in ppm using a solvent resonance as an internal standard. Data are reported as follows: Chemical shift, integration, multiplicity (s=singlet, d=doublet, t=triplet, m=multiplet, bs=broad singlet, bt=broad triplet, dd=doublet of a doublet) and coupling constants. Dichloromethane (DCM) was dried using activated alumina columns from Solv-Tek (Berryville, Va.). Dimethyl formamide (DMF) was dried by distillation over CaH₂ under N₂. All other commercially obtained reagents were used as received. Medium pressure liquid chromatography (MPLC) was performed with Biotage SP-1 (Charlottesville, Va.) using silica gel 60 (EM Science, 230-400 mesh).

### D. Formulation of Pharmaceutical Compositions

The pharmaceutical compositions provided herein contain therapeutically effective amounts of one or more of the compounds provided herein that are useful in the treatment, prevention, or amelioration of one or more of the symptoms associated with JNK1, JNK2, or JNK3 activity, or a disorder, condition, or ailment in which JNK1, JNK2, or JNK3 activity (e.g., type-2 diabetes, rheumatoid arthritis, Parkinson's disease, multiple sclerosis, and Alzheimer's disease) is implicated, and a pharmaceutically acceptable carrier. Pharmaceutical carriers suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. For example, the compounds may be formulated or combined with known NSAIDs, anti-inflammatory compounds, steroids, and/or antibiotics.

The compositions contain one or more compounds provided herein. The compounds are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (See, e.g., Ansel, Introduction to Pharmaceutical Dosage Forms, 4th Edition, 1985, 126).

In the compositions, effective concentration(s) of one or more compounds or pharmaceutically acceptable salts thereof is (are) mixed with a suitable pharmaceutical carrier. The concentrations of the compounds in the compositions are effective for delivery of an amount, upon administration, that treats, prevents, or ameliorates one or more of the symptoms of JNK1, JNK2, or JNK3 activity.

In one embodiment, the compositions are formulated for single dosage administration. To formulate a composition, the weight fraction of compound is dissolved, suspended, dispersed or otherwise mixed in a selected carrier at an effective concentration such that the treated condition is relieved or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro, ex vivo and in vivo systems, and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art.

Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disorder being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable salts thereof The pharmaceutically therapeutically active compounds and salts thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

Actual methods of preparing such dosage fauns are known, or will be apparent, to those skilled in this art; for example, See Remington's Pharmaceutical Sciences, Mack Publishing Company, Easton, Pa., 15th Edition, 1975.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, or in one embodiment 0.1-95%.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

a. Solid Compositions for Oral Administration

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or a pharmaceutically acceptable salt thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action. The active ingredient is a compound or pharmaceutically acceptable salt thereof as described herein. Higher concentrations, up to about 98% by weight of the active ingredient, may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

b. Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

2. Injectables, Solutions, and Emulsions

Parenteral administration, in one embodiment characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In, addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (See, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include sodium chloride injection, ringers injection, isotonic dextrose injection, sterile water injection, dextrose and lactated ringers injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration should be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable salts thereof may be formulated as aerosols for topical application, such as by inhalation (See, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5. Compositions for Other Routes of Administration

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 g.

Tablets and capsules for rectal administration are manufactured using the same pharmaceutically acceptable substance and by the same methods as for formulations for oral administration.

6. Targeted Formulations

The compounds provided herein, or pharmaceutically acceptable salts thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, See, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline (PBS) lacking divalent cations is added and the flask shaken until the lipid film is dispersed. The resulting vesicles are washed to remove unencapsulated compound, pelleted by centrifugation, and then resuspended in PBS.

7. Articles of Manufacture

The compounds or pharmaceutically acceptable salts may be packaged as articles of manufacture (e.g., kits) containing packaging material, a compound or pharmaceutically acceptable salt thereof provided herein within the packaging material, and a label that indicates that the compound or composition, or pharmaceutically acceptable salt thereof, is useful for treatment, prevention, or amelioration of one or more symptoms or disorders in which JNK1, JNK2, or JNK3 activity, including type-2 diabetes, rheumatoid arthritis, Parkinson's disease, multiple sclerosis, and Alzheimer's disease is implicated.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment.

8. Sustained Release Formulations

Also provided are sustained release formulations to deliver the compounds to the desired target at high circulating levels (between 10⁻⁹ and 10⁻⁴ M). The levels are either circulating in the patient systemically, or in one embodiment, localized to a site of, e.g., paralysis.

It is understood that the compound levels are maintained over a certain period of time as is desired and can be easily determined by one skilled in the art. Such sustained and/or timed release formulations may be made by sustained release means of delivery devices that are well known to those of ordinary skill in the art, such as those described in U.S. Pat. Nos. 3,845,770; 3,916,899; 3,536,809; 3, 598,123; 4,008,719; 4,710,384; 5,674,533; 5,059,595; 5,591,767; 5,120,548; 5,073,543; 5,639,476; 5,354,556 and 5,733,566, the disclosures of which are each incorporated herein by reference. These pharmaceutical compositions can be used to provide slow or sustained release of one or more of the active compounds using, for example, hydroxypropylmethyl cellulose, other polymer matrices, gels, permeable membranes, osmotic systems, multilayer coatings, microparticles, liposomes, microspheres, or the like. Suitable sustained release formulations known to those skilled in the art, including those described herein, may be readily selected for use with the pharmaceutical compositions provided herein. Thus, single unit dosage forms suitable for oral administration, such as, but not limited to, tablets, capsules, gelcaps, caplets, powders and the like, that are adapted for sustained release are contemplated herein.

In one embodiment, the sustained release formulation contains active compound such as, but not limited to, microcrystalline cellulose, maltodextrin, ethylcellulose, and magnesium stearate. As described above, all known methods for encapsulation which are compatible with properties of the disclosed compounds are contemplated herein. The sustained release formulation is encapsulated by coating particles or granules of the pharmaceutical compositions provided herein with varying thickness of slowly soluble polymers or by microencapsulation. In one embodiment, the sustained release formulation is encapsulated with a coating material of varying thickness (e.g. about 1 micron to 200 microns) that allow the dissolution of the pharmaceutical composition about 48 hours to about 72 hours after administration to a mammal. In another embodiment, the coating material is a food-approved additive.

In another embodiment, the sustained release formulation is a matrix dissolution device that is prepared by compressing the drug with a slowly soluble polymer carrier into a tablet. In one embodiment, the coated particles have a size range between about 0.1 to about 300 microns, as disclosed in U.S. Pat. Nos. 4,710,384 and 5,354,556, which are incorporated herein by reference in their entireties. Each of the particles is in the form of a micromatrix, with the active ingredient uniformly distributed throughout the polymer.

Sustained release formulations such as those described in U.S. Pat. No. 4,710,384, which is incorporated herein by reference in its entirety, having a relatively high percentage of plasticizer in the coating in order to permit sufficient flexibility to prevent substantial breakage during compression are disclosed. The specific amount of plasticizer varies depending on the nature of the coating and the particular plasticizer used. The amount may be readily determined empirically by testing the release characteristics of the tablets formed. If the medicament is released too quickly, then more plasticizer is used. Release characteristics are also a function of the thickness of the coating. When substantial amounts of plasticizer are used, the sustained release capacity of the coating diminishes. Thus, the thickness of the coating may be increased slightly to make up for an increase in the amount of plasticizer. Generally, the plasticizer in such an embodiment will be present in an amount of about 15 to 30% of the sustained release material in the coating, in one embodiment 20 to 25%, and the amount of coating will be from 10 to 25% of the weight of the active material, and in another embodiment, 15 to 20% of the weight of active material. Any conventional pharmaceutically acceptable plasticizer may be incorporated into the coating.

The compounds provided herein can be formulated as a sustained and/or timed release formulation. All sustained release pharmaceutical products have a common goal of improving drug therapy over that achieved by their non-sustained counterparts. Ideally, the use of an optimally designed sustained release preparation in medical treatment is characterized by a minimum of drug substance being employed to cure or control the condition. Advantages of sustained release formulations may include: 1) extended activity of the composition, 2) reduced dosage frequency, and 3) increased patient compliance. In addition, sustained release formulations can be used to affect the time of onset of action or other characteristics, such as blood levels of the composition, and thus can affect the occurrence of side effects.

The sustained release formulations provided herein are designed to initially release an amount of the therapeutic composition that promptly produces the desired therapeutic effect, and gradually and continually release of other amounts of compositions to maintain this level of therapeutic effect over an extended period of time. In order to maintain this constant level in the body, the therapeutic composition must be released from the dosage form at a rate that will replace the composition being metabolized and excreted from the body.

The sustained release of an active ingredient may be stimulated by various inducers, for example pH, temperature, enzymes, water, or other physiological conditions or compounds.

Preparations for oral administration may be suitably formulated to give controlled release of the active compound. In one embodiment, the compounds are formulated as controlled release powders of discrete microparticles that can be readily formulated in liquid form. The sustained release powder comprises particles containing an active ingredient and optionally, an excipient with at least one non-toxic polymer.

The powder can be dispersed or suspended in a liquid vehicle and will maintain its sustained release characteristics for a useful period of time. These dispersions or suspensions have both chemical stability and stability in terms of dissolution rate. The powder may contain an excipient comprising a polymer, which may be soluble, insoluble, permeable, impermeable, or biodegradable. The polymers may be polymers or copolymers. The polymer may be a natural or synthetic polymer. Natural polymers include polypeptides (e.g., zein), polysaccharides (e.g., cellulose), and alginic acid. Representative synthetic polymers include those described, but not limited to, those described in column 3, lines 33-45 of U.S. Pat. No. 5,354,556, which is incorporated by reference in its entirety. Particularly suitable polymers include those described, but not limited to those described in column 3, line 46-column 4, line 8 of U.S. Pat. No. 5,354,556 which is incorporated by reference in its entirety.

The sustained release compositions provided herein may be formulated for parenteral administration, e.g., by intramuscular injections or implants for subcutaneous tissues and various body cavities and transdermal devices. In one embodiment, intramuscular injections are formulated as aqueous or oil suspensions. In an aqueous suspension, the sustained release effect is due to, in part, a reduction in solubility of the active compound upon complexation or a decrease in dissolution rate. A similar approach is taken with oil suspensions and solutions, wherein the release rate of an active compound is determined by partitioning of the active compound out of the oil into the surrounding aqueous medium. Only active compounds which are oil soluble and have the desired partition characteristics are suitable. Oils that may be used for intramuscular injection include, but are not limited to, sesame, olive, arachis, maize, almond, soybean, cottonseed and castor oil.

A highly developed form of drug delivery that imparts sustained release over periods of time ranging from days to years is to implant a drug-bearing polymeric device subcutaneously or in various body cavities. The polymer material used in an implant, which must be biocompatible and nontoxic, include but are not limited to hydrogels, silicones, polyethylenes, ethylene-vinyl acetate copolymers, or biodegradable polymers.

### E. Evaluation of the Activity of the Compounds

The activity of the compounds provided herein as inhibitors of JNK1, JNK2, or JNK3 activity may be measured in standard assays, e.g., X-ray crystallographic analysis of inhibitor-bound JNK1, JNK2, or JNK3 complexes, enzymatic inhibition assays, cell cytoprotection and viability assays (as described below).

A number of potential inhibitors were tested for selectivity against JNK1, JNK2, or JNK3 and potency was compared with the non-selective JNK inhibitor SP600125. The optimal concentration of the non-selective JNK inhibitor SP600125 for inhibition of JNKs was initially determined. Active JNK1, active JNK2 or active JNK3 was incubated with His-c-Jun (1-201) as substrate and [gamma-³²P]ATP either in the absence or presence of increasing doses of SP600125. The ³²P labeled c-Jun bands were visualized by autoradiography. As expected, SP600125 effectively inhibited 80% activity of JNK1, JNK2, and JNK3 in vitro at a 10 μM concentration. Therefore, this concentration was chosen for comparison with a 10 μM concentration of each potential inhibitor. Further, SP600125 showed no selectivity for JNK1, JNK2 or JNK3, inhibiting each equally.

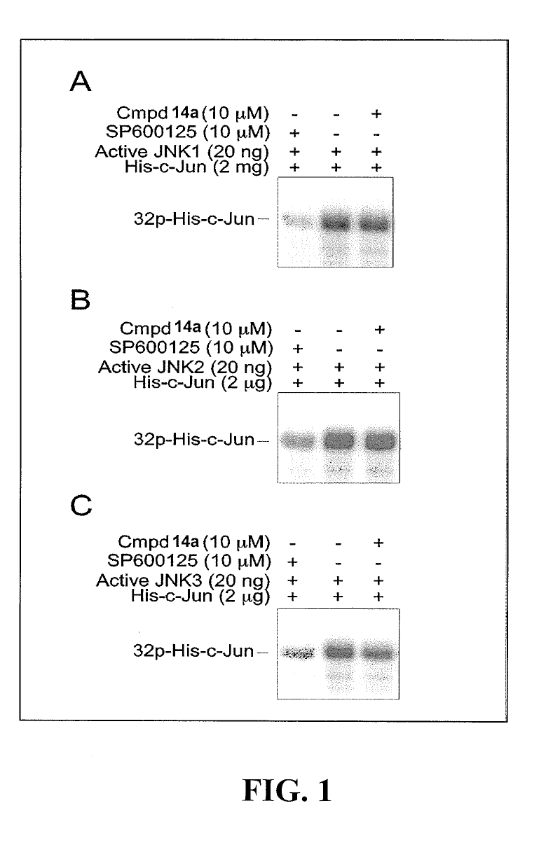

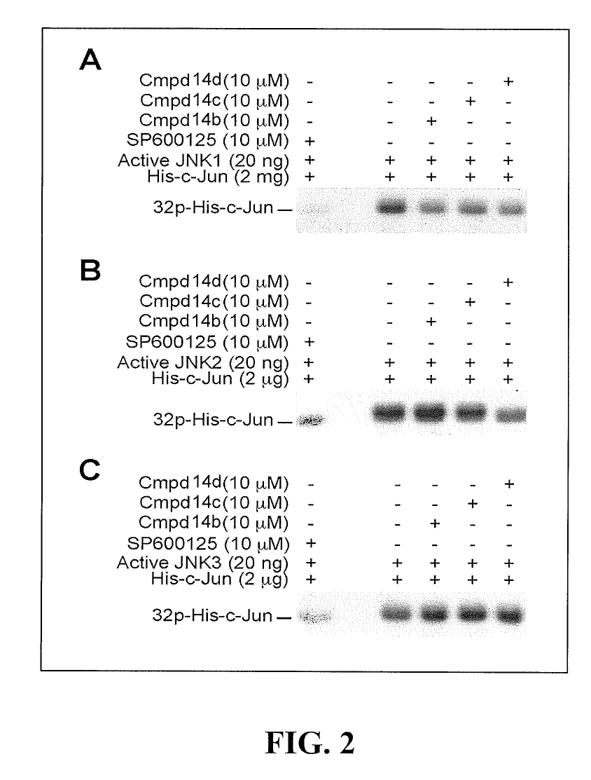

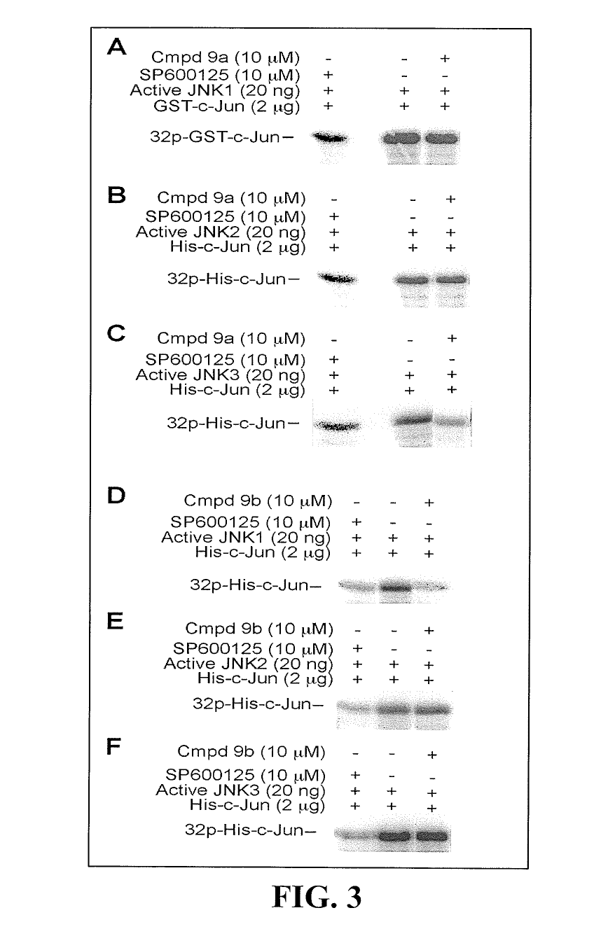

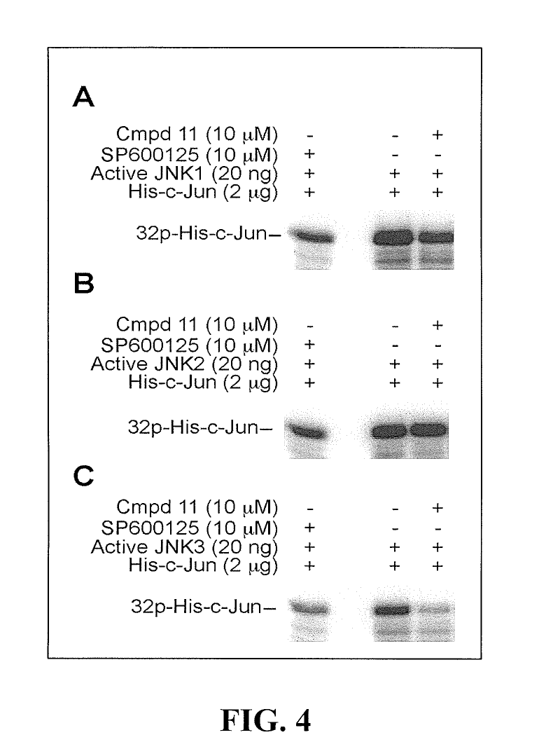

A number of candidate compounds including 14a-d, 9a-b, and 11 were tested for specificity and potency against JNK1, JNK2, and JNK3 activity compared to SP600125 in an in vitro kinase assay. Active JNK1, active JNK2, or active JNK3 was incubated with His-c-Jun (1-201), as substrate, [gamma-³²P]ATP, and 10 micromolar of each new compound. SP600125 (10 μM) was used as an internal control. The ³²P labeled c-Jun bands were resolved by SDS-PAGE and visualized by autoradiography. Compound 14a (FIG. 1) inhibited JNK3, but not JNK1 or JNK2, by about 50% at a concentration of 10 μM. Therefore, 14a is selective for JNK3 activity. Compound 14b (FIG. 2) inhibited JNK1 activity, but not JNK2 or JNK3. Compound 14c showed a weak inhibitory effect on JNK1 and a very weak effect on JNK2 activity. Compound 14d inhibited JNK1 and JNK2 activity together. Overall, 14b had a selective inhibitory effect on JNK1. Compound 9a (FIG. 3) inhibited JNK3 activity, but not JNK1 or JNK2. Compound 9b inhibited JNK1 activity, but not JNK2 or JNK3. Compound 11 (FIG. 4) inhibited JNK3 activity, but not JNK1 or JNK2. To examine the inhibition of 14b on JNK1 activity in more detail, an in vitro kinase assay was conducted with various doses of 14b. The inhibitory effect of different doses of 14b on JNK1 was measured. Active JNK1 was incubated with His-c-Jun (1-201) as substrate, [gamma-³²P]ATP and various doses of 14b as indicated. The results indicated that 25 μM 14b inhibited JNK1 activity by about 50% in vitro.

The effectiveness of the JNK1 inhibitor 14b against a newly discovered JNK1 substrate, Myt1was also tested. The pcDNA3-V5-JNK1 plasmid was co-transfected with pcDNA3-myc-Myt1 into HEK293 cells and then cultured for 36 h at 37° C. in 5% CO₂ incubator. Cells transfected with the pcDNA3-mock vector served as negative control. Cells were treated or not treated for 12 h with 1, 5, or 10 μM of the JNK1 inhibitor 14b to determine a dose response and then the proteins were extracted and used for immunoprecipiation (IP) with anti-V5. Myt1 was visualized by immunoblot with anti-Myc horseradish peroxidase. For visualizing the exogenous Myt1, JNK1, or phosphorylation of c-Jun, total cell lysates were used for immunoblotting with anti-Myc-HRP, -V4, or -phospho-c-Jun. c-Jun was used as a positive control and results indicated that the inhibitor effectively blocked the binding of JNK1 with Myt1. The effect of the JNK1 inhibitor, 14b, on the ex vivo regulation of UVA-induced JNK1 binding with Myt1 was then examined. Human melanoma SK-MEL-28 cells were seeded and cultured for 24 h in 10% FBS/MEM in a 37° C., 5% CO₂ incubator. The cells were then serum-deprived for 24 h, pretreated or not pretreated with the JNK1 specific inhibitor, 14b exposed or not exposed to UVA (40 kJ/m²), and harvested after incubation for 6 h. Immunoprecipitation (IP) was performed to precipitate endogenous JNK1 and then the endogenous Myt1 protein was detected with anti-Myt1. The immunoprecipitated active JNK1 protein was incubated with GSTc-Jun for 60 min at 30° C. for an in vitro kinase assay and the ³²P-labeled c-Jun was visualized by autoradiography. These results further confirmed the specificity of the JNK1 inhibitor.

### F. Methods of Use

Methods used to test the effectiveness and specificity of JNK1 and JNK3 inhibitors have been described.

In one aspect, the invention provides a method of modulating (such as, inhibiting) an activity of JNK1 including, contacting JNK1 with a compound of Formula I, or a pharmaceutically acceptable salt thereof.

In some embodiments, a compound of Formula I, or a pharmaceutically acceptable salt thereof, is a selective inhibitor of JNK1 over JNK2 and JNK3.

In some embodiments, a selective inhibitor of JNK1 over JNK2 and JNK3 is a compound of Formula IV:

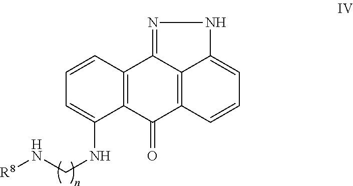

wherein:

(i) R⁸═C₆H₅, n=6; or

(ii) R⁸═C₆H₅, n=7; or

(iii) R⁸═C₆H₅C(O), n=9; or a pharmaceutically acceptable salt thereof.

In some embodiments, a selective inhibitor of JNK1 over JNK2 and JNK3 is a compound of Formula IV:

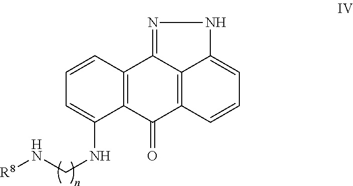

wherein:

(i) R⁸═C₆H₅, n=4-9; or

(ii) R⁸═C₆H₅C(O), n=9; or a pharmaceutically acceptable salt thereof.

In another aspect, the invention provides a method of modulating (such as, inhibiting) an activity of JNK3 including, contacting JNK3 with a compound of Formula I, or a pharmaceutically acceptable salt thereof.

In some embodiments, a compound of Formula I, or a pharmaceutically acceptable salt thereof, is a selective inhibitor of JNK3 over JNK1 and JNK2.

In some embodiments, a selective inhibitor of JNK3 over JNK1 and JNK2 is a compound of Formula V:

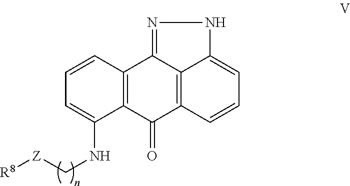

wherein:

(i) R⁸═C₆H₅, Z═NH, n=4-9; or

(ii) R⁸=4-CH₃—C₆H₄, Z═O, n=4-9; or

(iii) R⁸═C₆H₅C(O), Z═NH, n=4-9.

In some embodiments, a selective inhibitor of JNK3 over JNK1 and JNK2 is a compound of Formula V:

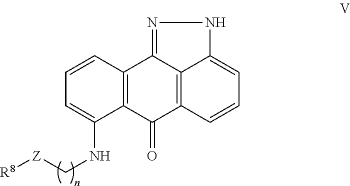

wherein:

(i) R⁸═C₆H₅, Z═NH, n=5; or

(ii) R⁸=4-CH₃—C₆H₄, Z═O, n=5; or

(iii) R⁸═C₆H₅C(O), Z═NH, n=7; or a pharmaceutically acceptable salt thereof.

In another aspect, the invention provides a method of modulating (such as, inhibiting) an activity of JNK1 and JNK2 including, contacting JNK1 and JNK2 with a compound of Formula I, or a pharmaceutically acceptable salt thereof.

In some embodiments, a compound of Formula I, or a pharmaceutically acceptable salt thereof, is a selective inhibitor of JNK1 and JNK2 over JNK3.

In some embodiments, a selective inhibitor of JNK1 and JNK2 over JNK3 is a compound of Formula V:

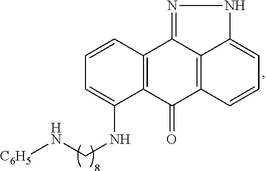

or a pharmaceutically acceptable salt thereof.

In some embodiments, a selective inhibitor of JNK1 and JNK2 over JNK3 is a compound of Formula VI:

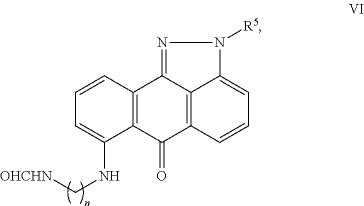

wherein, n=3-10; or a pharmaceutically acceptable salt thereof.

In some embodiments, a selective inhibitor of JNK1 and JNK2 over JNK3 is a compound of Formula VI:

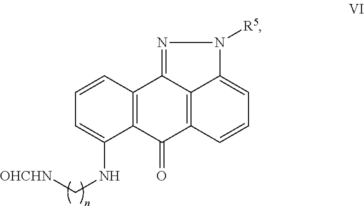

wherein, n=5, 6, 7, or 8; or a pharmaceutically acceptable salt thereof.

In some embodiments, a selective inhibitor of JNK1 and JNK2 over JNK3 is:

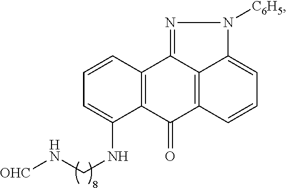

or a pharmaceutically acceptable salt thereof.

Also described are methods for treating, preventing, or ameliorating one or more symptoms, disorders, or conditions associated with JNK1, JNK2, or JNK3 activity in a mammal (e.g., a human). The methods can employ a composition comprising any of the compounds provided herein.

In another aspect, the invention provides a method for treating, preventing, or ameliorating one or more symptoms associated with type-2 diabetes including, administering to a subject in need thereof a therapeutically effective amount of a compound of Formula I, or a pharmaceutically acceptable salt thereof.

In some embodiments, the compound of Formula I is a compound of Formula IV:

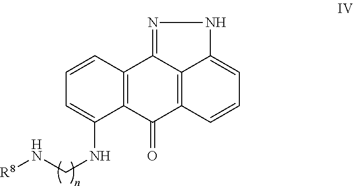

wherein:

(i) R⁸═C₆H₅, n=6; or

(ii) R⁸═C₆H₅, n=7; or

(iii) R⁸═C₆H₅C(O), n=9; or a pharmaceutically acceptable salt thereof.

In another aspect, the invention provides a method for treating, preventing, or ameliorating one or more symptoms associated with insulin resistance including, administering to a subject in need thereof a therapeutically effective amount of a compound of Formula I, or a pharmaceutically acceptable salt thereof.

In another aspect, the invention provides a method for treating, preventing, or ameliorating one or more symptoms associated with neural degeneration (such as, Parkinson's disease, multiple sclerosis, or Alzheimer's disease) including, administering to a subject in need thereof a therapeutically effective amount of a compound of Formula I, or a pharmaceutically acceptable salt thereof.

In some embodiments, the compound of Formula I is a compound of Formula V:

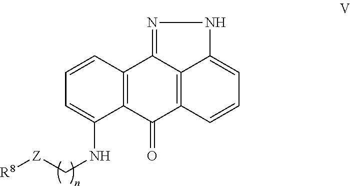

wherein:

(i) R⁸═C₆H₅, Z═NH, n=5; or

(ii) R⁸=4-CH₃—C₆H₄, Z═O, n=5; or

(iii) R⁸═C₆H₅C(O), Z═NH, n=7; or a pharmaceutically acceptable salt thereof.

In another aspect, the invention provides a method for treating, preventing, or ameliorating one or more symptoms associated with rheumatoid arthritis including, administering to a subject in need thereof a therapeutically effective amount of a compound of Formula I, or a pharmaceutically acceptable salt thereof

In some embodiments, the compound of Formula I is:

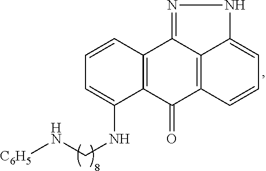

or a pharmaceutically acceptable salt thereof.

In some embodiments, the compound of Formula I is a compound of Formula VI:

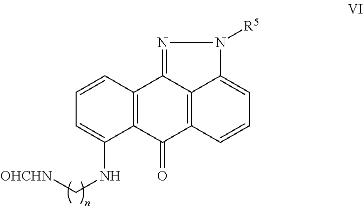

wherein:

n=3-10; or a pharmaceutically acceptable salt thereof.

In some embodiments, the compound of Formula I is:

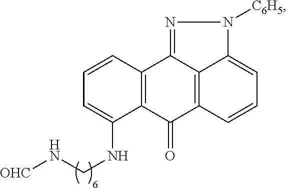

or a pharmaceutically acceptable salt thereof.

### G. Methods of Designing Inhibitors Targeting the Activation Loop of JNKs1-3

Provided herein are methods, including computer-based methods, for designing compounds that bind to and/or inhibit an activation-loop of the JNK1, JNK2, or JNK3 as set forth in the three-dimensional model structures.

The inventors have determined that the conformations of residues in the activation-loop of the JNK1, JNK2, or JNK3 are useful for determining inhibitors with high affinity for the active site. Thus, given the three-dimensional model described herein as well as the activation-loop of the JNK1, JNK2, or JNK3 as useful residues to target, one having ordinary skill in the art would know how to use standard molecular modeling or other techniques to identify peptides, peptidomimetics, and small-molecules that would bind to or interact with one or more of the residues in activation-loop of the JNK1, JNK2, or JNK3.

By “molecular modeling” is meant quantitative and/or qualitative analysis of the structure and function of physical interactions based on three-dimensional structural information and interaction models. This includes conventional numeric-based molecular dynamic and energy minimization models, interactive computer graphic models, modified molecular mechanics models, distance geometry and other structure-based constraint models. Molecular modeling typically is performed using a computer and may be further optimized using known methods.

Methods of designing compounds that bind specifically (e.g., with high affinity) to one or more of the residues described previously typically are also computer-based, and involve the use of a computer having a program capable of generating an atomic model. Computer programs that use X-ray crystallography data or molecular model coordinate data, such as the data that are available from the PDB, are particularly useful for designing such compounds. Programs such as RasMol, for example, can be used to generate a three dimensional model. Computer programs such as INSIGHT (Accelrys, Burlington, Mass.), Auto-Dock (Accelrys), and Discovery Studio 1.5 (Accelrys) allow for further manipulation and the ability to introduce new structures.

Compounds can be designed using, for example, computer hardware or software, or a combination of both. However, designing is preferably implemented in one or more computer programs executing on one or more programmable computers, each containing a processor and at least one input device. The computer(s) preferably also contain(s) a data storage system (including volatile and non-volatile memory and/or storage elements) and at least one output device. Program code is applied to input data to perform the functions described above and generate output information. The output information is applied to one or more output devices in a known fashion. The computer can be, for example, a personal computer, microcomputer, or work station of conventional design.

Each program is preferably implemented in a high level procedural or object oriented programming language to communicate with a computer system. However, the programs can be implemented in assembly or machine language, if desired. In any case, the language can be a compiled or interpreted language.

Each computer program is preferably stored on a storage media or device (e.g., ROM or magnetic diskette) readable by a general or special purpose programmable computer. The computer program serves to configure and operate the computer to perform the procedures described herein when the program is read by the computer. The method of the invention can also be implemented by means of a computer-readable storage medium, configured with a computer program, where the storage medium so configured causes a computer to operate in a specific and predefined manner to perform the functions described herein.

For example, a computer-assisted method of generating a test inhibitor of the activation-loop of JNK1, JNK2, and JNK3 as set forth by the three-dimensional computational structure is provided. The method uses a programmed computer comprising a processor and an input device, and can include:

(a) inputting on the input device, e.g., through a keyboard, a diskette, or a tape, data (e.g. atomic coordinates) comprising a docking box surrounded by one or more residues of the activation-loop of JNK1, JNK2, and JNK3 as defined by the three-dimensional computational structure;

(b) docking into the docking box a test inhibitor molecule using the processor; and

(c) determining, based on the docking, whether the test inhibitor molecule would be capable of interacting with the one or more residues of the active site.

By “capable of interacting” it is meant capable of forming one or more hydrogen bonds, ionic bonds, covalent bonds, pi-pi interactions, cation-pi interactions, sulfur-aromatic interactions, or van der Waals interactions. In some embodiments, the test inhibitor molecule can interact with one or more residues (e.g., one or more residues of region I or II) of the activation-loop of JNK1, JNK2, and JNK3 with a minimum interaction energy of −5 to about −50 kcal/mol, e.g., −20 to −40 kcal/mol. In some embodiments, the test inhibitor would be capable of forming a hydrogen bond with one or more residues of the activation-loop of JNK1, JNK2, and JNK3.

The inhibitory activity of the test inhibitor on JNK1, JNK2 and JNK3 in vitro can be evaluated. In some embodiments, the inhibitory activity is evaluated using a kinase assay (see Example 8).

From the information obtained using these methods, one skilled in the art will be able to design and make inhibitory compounds (e.g., peptides, non-peptide small molecules, peptidomimetics, and aptamers (e.g., nucleic acid aptamers)) with the appropriate 3-D structure, e.g., at certain residues and that interact in certain manners (e.g., hydrogen-bonding, ion bonding, covalent bonding, pi-pi interactions, sulfur-aromatic interactions, steric interactions, and/or van der Waals interactions).

Moreover, if computer-usable 3-D data (e.g., x-ray crystallographic data) for a candidate compound are available, one or more of the following computer-based steps can be performed in conjunction with computer-based steps described above:

(c) inputting into an input device, e.g., through a keyboard, a diskette, or a tape, data (e.g. atomic coordinates) that define the three-dimensional (3-D) structure of a candidate compound;

(d) determining, using a processor, the 3-D structure (e.g., an atomic model) of the candidate compound;

(e) determining, using the processor, whether the candidate compound binds to or interacts with one or more of the residues of interest in the activation-loop of JNK1, JNK2, and JNK3;

(f) determining the interaction energy of the candidate compound; and

(g) identifying the candidate compound as a compound that inhibits the site.

The method can involve an additional step of outputting to an output device a model of the 3-D structure of the compound. In addition, the 3-D data of candidate compounds can be compared to a computer database of, for example, 3-D structures stored in a data storage system. In some embodiments, the interaction energy of the candidate compound is less than −54 kcal/mol.

Candidate compounds identified as described above can then be tested in standard cellular inhibition assays familiar to those skilled in the art.

The 3-D structure of molecules can be determined from data obtained by a variety of methodologies. These methodologies include: (a) x-ray crystallography; (b) nuclear magnetic resonance (NMR) spectroscopy; (c) molecular modeling methods, e.g., homology modeling techniques, threading algorithms, and in particular the refined homology modeling methods.

Any available method can be used to construct a 3-D model of the JNKs activation loop site from the x-ray crystallographic, molecular modeling, and/or NMR data using a computer as described above. Such a model can be constructed from analytical data points inputted into the computer by an input device and by means of a processor using known software packages, e.g., CATALYST (Accelrys), INSIGHT (Accelrys) and CeriusII, HKL, MOSFILM, XDS, CCP4, SHARP, PHASES, HEAVY, XPLOR, TNT, NMRCOMPASS, NMRPIPE, DIANA, NMRDRAW, FELIX, VNMR, MADIGRAS, QUANTA, BUSTER, SOLVE, FRODO, or CHAIN. The model constructed from these data can be visualized via an output device of a computer, using available systems, e.g., Silicon Graphics, Evans and Sutherland, SUN, Hewlett Packard, Apple Macintosh, DEC, IBM, or Compaq.

Once the 3-D structure of a compound that binds to or interacts with one or more residues of the activation-loop of the JNK1, JNK2, and JNK3 have been established using any of the above methods, a compound that has substantially the same 3-D structure (or contains a domain that has substantially the same structure) as the identified compound can be made. In this context, “has substantially the same 3-D structure” means that the compound possesses a hydrogen bonding and hydrophobic character that is similar to the identified compound. In some cases, a compound having substantially the same 3-D structure as the identified compound can include a hydroxyl or alkyl moiety.

With the above described 3-D structural data in hand and knowing the chemical structure (e.g., amino acid sequence in the case of a protein) of the region of interest, those of skill in the art would know how to make compounds with the above-described properties. Moreover, one having ordinary skill in the art would know how to derivatize such compounds. Such methods include chemical synthetic methods and, in the case of proteins, recombinant methods.

While not essential, computer-based methods can be used to design the compounds of the invention. Appropriate computer programs include: InsightII (Accelrys), CATALYST (Accelrys), LUDI (Accelrys., San Diego, Calif.), Aladdin (Daylight Chemical Information Systems, Irvine, Calif.); and LEGEND [Nishibata et al. (1985) J. Med. Chem. 36(20):2921-2928], as well as the methods described in the Examples below and the references cited therein.

The invention is further described in the following examples, which do not limit the scope of the invention described in the claims.

### EXAMPLES

### Example 1

### 7-(5-N-Phenylaminopentyl)-2H-anthra[1,9-cd]pyrazol-6-one (5a)

### Step 1. 7-Chloro-2H-anthra[1,9-cd]pyrazol-6-one (2):

To a stirred solution of 1,5-dichloroanthraquinone 1 (Sigma Aldrich Chemicals (St. Louis, Mo.), 1.00 g, 3.61 mmol) in pyridine (8.75 mL) was added hydrazine monohydrate (0.25 g, 4.99 mmol) and the reaction mixture was refluxed for 16 h. The resultant mixture was cooled to room temperature and the solvent was removed under vacuum. MPLC purification of the residue (CHCl₃:MeOH 99:1) gave 2 (85%) as an amorphous yellow solid; mp 310° C. ¹H NMR (400 MHz, DMSO-d₆): δ 7.55 (1H, d, J=8.0 Hz), 7.64-7.72 (2H, m), 7.82 (1H, d, J=7.8 Hz), 7.93 (1H, d, J=8.0 Hz), 8.16 (1H, d, J=7.8 Hz) and 13.80 (1H, s).

### Step 2. 7-(5-Aminopentyl)amino-2H-anthra[1,9-cd]pyrazol-6-one (3a)

To a stirred solution of 2 (0.25 g, 1.00 mmol) in DMSO (5 mL) was added 1,5-diaminopentane (0.61 g, 6.00 mmol) and the reaction mixture was refluxed for 4 h. The resultant mixture was cooled to room temperature and was partitioned between chloroform and water. The aqueous layer was extracted with chloroform (3×20 mL). The combined organic layer was dried over anhydrous sodium sulfate, filtered and concentrated under vacuum. MPLC purification (CHCl₃:MeOH:NH₄OH 94:5:1) of the residue gave compound 3a (0.24 g, 76% yield) as an amorphous red solid. ¹H NMR (400 MHz, CD₃OD): δ 1.58-1.71 (4H, m), 1.81-1.84 (2H, m), 2.89 (2H, m), 3.38 (2H, t, J=7.2 Hz), 6.85 (1H, d, J=8.0 Hz), 7.45-7.51 (2H, m), 7.64-7.66 (1H, m), 7.77 (1H, d, J=9.0 Hz) and 7.84 (1H, m).

### Step 3. 7-(5-N-Phenylaminopentyl)-2H-anthra[1,9-cd]pyrazol-6-one (5a)

An oven-dried pyrex screw tube was charged with CsOAc (90 mg, 0.47 mmol) and CuI (36 mg, 0.19 mmol). The tube was evacuated and backfilled with nitrogen. To this mixture were added dry benzene (0.3 mL), degassed DMF (6 mL), iodobenzene (20.9 μL, 0.19 mmol), compound 3a (0.12 g, 0.38 mmol). The reaction mixture was stirred at 90° C. for 16 h. To the resultant mixture was added saturated NaHCO₃ solution (15 mL) and ethyl acetate. The aqueous layer was washed with ethyl acetate (3×20 mL). The combined organic layer was dried over anhydrous sodium sulfate, filtered and concentrated under vacuum. MPLC purification (CHCl₃:MeOH 99:1) of the residue gave 5a (30 mg, 20% yield) as an amorphous red solid; mp 151-152° C. The compound 5a was further purified using HPLC [Zorbax CN, 5 μm, 21×250 mm, eluting with linear gradient of 20% to 50% of isopropanol in hexanes over 40 minutes and flow rate of 10.0 mL/min with a retention time of 25.6 minutes for 5a]. ¹H NMR (400 MHz, CDCl₃): δ 1.50-1.62 (4H, m), 1.76-1.84 (2H, m), 3.26-3.32 (4H, m), 5.78 (1H, bs), 6.78 (1H, d, J=8.0 Hz), 7.38 (1H, m), 7.54-7.60 (3H, m), 7.66-7.74 (2H, m), 7.86 (2H, d, J=7.8 Hz), 7.90-7.96 (2H, m), 8.21 (1H, s) and 10.0.8 (1H, bt).

### Example 2

### 7-(6-N-Phenylaminohexyl)amino-2H-anthra[1,9-cd]pyrazol-6-one (5b)

### Step 1. 2-N-Boc-7-(6-N-Boc-Aminohexyl)amino-2H-anthra[1,9-cd]pyrazol-6-one (3b)

To a stirred solution of 2 (0.25 g, 1.00 mmol) in pyridine (5 mL) was added 1,6-diaminohexane (0.58 g, 5.00 mmol) and the reaction mixture was refluxed for 16 h. The resultant mixture was cooled to room temperature, partitioned between chloroform and water. The aqueous layer was extracted with chloroform (3×20 mL). The combined organic layer was dried over anhydrous sodium sulfate, filtered and concentrated under vacuum. The red colored residue was subjected to the next reaction without purification. To the residue (0.40 g, 1.20 mmol) in dichloromethane was added triethylamine (1.0 mL, 7.20 mmol) and BOC anhydride (1.80 g, 6.89 mmol). The reaction mixture was stirred for 16 h at room temperature. The solvent was removed by evaporation in vacuo. MPLC purification (hexanes:dichloromethane 70:30) of the residue gave compound 3b (0.24 g, 46% yield) as a red solid. ¹H NMR (400 MHz, CDCl₃): δ 1.44 (11H, m), 1.50 (4H, m), 1.78-1.82 (11H, m), 3.10 (2H, m), 3.28 (2H, m), 4.60 (1H, bs), 6.82 (1H, d, J=7.8 Hz), 7.44 (1H, m), 7.62 (2H, m), 7.94 (1H, d, J=7.80 Hz) and 9.98 (1H, bt).

### Step 2. 7-(6-Aminohexyl)amino-2H-anthra[1,9-cd]pyrazol-6-one TFA salt (4b)

To a stirred solution of 3b (0.24 g, 0.55 mmol) in dichloromethane was added trifluoroacetic acid (4 mL) and stirred at room temperature for 12 h. The solvent was removed under vacuum to give 4b (0.23 g) as an amorphous red solid. ¹H NMR (400 MHz, CD₃OD): δ 1.50 (2H, m), 1.58 (2H, m), 1.68 (2H, m), 1.74 (2H, m), 2.92 (2H, m), 3.28 (2H, m), 6.76 (1H, d, J=8.0 Hz), 7.34 (1H, d, J=7.6 Hz), 7.41 (1H, m), 7.58 (1H, m), 7.79 (1H, d, J=7.6 Hz) and 7.98 (1H, d, J=8.0 Hz).

### Step 3. 7-(6-N-Phenylaminohexyl)amino-2H-anthra[1,9-cd]pyrazol-6-one (5b)

The compound 5b (60 mg, 8% yield) was prepared from 4b (0.60 g, 1.79 mmol) using the same procedure as 5a. HPLC purification of the above compound yielded 5b as an amorphous red solid; mp 158-161° C. HPLC conditions: Zorbax CN, 5 μm, 21×250 mm, eluting with linear gradient 20% to 80% of isopropanol in hexanes over 40 minutes, and flow rate of 10.0 mL/min with a retention time of 15.15 minutes for 5b. ¹H NMR (400 MHz, CDCl₃): δ 1.50 (2H, m), 1.58-1.62 (4H, m), 1.92-2.00 (2H, m), 3.30-3.34 (4H, m), 5.60 (1H, bs), 6.81 (1H, d, J=8.0 Hz), 7.38 (1H, m), 7.50 (1H, m), 7.56-7.68 (4H, m), 7.88 (2H, d, J=7.8 Hz), 7.94 (2H, m), 8.19 (1H, s) and 10.10 (1H, bt).

### Example 3

### 7-(7-N-Phenylaminoheptyl)amino-2H-anthra[1,9-cd]pyrazol-6-one (5c)

### Step 1. 2-N-Boc-7-(7-N-Boc-Aminoheptyl)amino-2H-anthra[1,9-cd]pyrazol-6-one (3c)

The compound 3c (0.41 g, 64% yield) was prepared from 2 (0.60 g, 1.80 mmol) and 1,7-diaminoheptane (1.63 g, 12.52 mmol) by using the same procedure as 3b. ¹H NMR (400 MHz, CDCl₃): 1.32-1.52 (17H, m), 1.70-1.80 (11H, m), 3.10 (2H, m), 3.32 (2H, m), 4.58 (1H, bs), 6.82 (1H, d, J=8.0 Hz), 7.44 (1H, m), 7.66 (2H, m), 7.98 (1H, d, J=8.0 Hz) and 10.0 (1H, bt).

### Step 2. 7-(7-Aminoheptyl)amino-2H-anthra[1,9-cd]pyrazol-6-one TFA salt (4c) (4c is a TFA salt of 6a)

The compound 4c (0.16 g) was prepared from 3c using the same procedure as 4b. ¹H NMR (400 MHz, DMSO-d₆): δ 1.24-1.38 (6H, m), 1.40-1.46 (2H, m), 1.62-1.70 (2H, m), 2.52 (2H, m), 3.58 (2H, m), 6.82 (1H, d, J=8.4 Hz), 7.36 (1H, d, J=6.8 Hz), 7.50 (1H, m), 7.64 (1H, m), 7.78 (1H, d, J=7.2 Hz), 7.80 (1H, d, J=8.0 Hz) and 10.0 (1H, bt).

### Step 3. 7-(7-N-Phenylaminoheptyl)amino-2H-anthra[1, 9-cd]pyrazol-6-one (5c)

The compound 5c (10 mg, 9% yield) was prepared from 4c 0.12 g, 0.26 mmol) using the same procedure as 5a. HPLC purification of the above compound yielded 5c as an amorphous red solid; mp 167-170° C. HPLC conditions: Zorbax CN, 5 μm, 21×250 mm, eluting with linear gradient 20% to 80% of isopropanol in hexanes over 40 minutes, and flow rate of 10.0 mL/min with a retention time of 15.10 minutes for 5c. ¹H NMR (400 MHz, CDCl₃): δ 1.30-1.37 (4H, m), 1.44-1.50 (4H, m), 1.68-1.74 (2H, m), 3.14-3.26 (4H, m), 5.70 (bs, 1H), 6.70 (1H, d, J=8.4 Hz), 7.30 (1H, m), 7.40 (1H, m), 7.48-7.58 (4H, m), 7.76-7.86 (4H, m), 8.21 (1H, s) and 9.89 (1H, bt).

### Example 4

### 7-(8-N-Phenylaminooctyl)amino-2H-anthra[1,9-cd]pyrazol-6-one (5d)

### Step 1. 2-N-Boc-7-(8-N-Boc-Aminooctyl)amino-2H-anthra[1,9-cd]pyrazol-6-one (3d)

The compound 3d (80 mg, 35% yield) was prepared from 2 (0.13 g, 0.50 mmol) and 1,8-diaminooctane (0.36 g, 2.50 mmol) by using the same procedure as 3b. ¹H NMR (400 MHz, CDCl₃): 1.32-1.52 (19H, m), 1.70-1.80 (11H, m), 3.10 (2H, m), 3.32 (2H, m), 4.58 (1H, bs), 6.82 (1H, d, J=8.0 Hz), 7.44 (1H, m), 7.66 (2H, m), 7.98 (1H, d, J=8.0 Hz) and 10.0 (1H, bt).

### Step 2. 7-(8-Aminooctyl)amino-2H-anthra[1,9-cd]pyrazol-6-one TFA salt (4d)

The compound 4d (80 mg) was prepared from 3d using the same procedure as 4b. ¹H NMR (400 MHz, DMSO-d₆): δ 1.30-1.50 (10H, m), 1.66 (2H, m), 2.50 (2H, m), 3.28 (2H, m), 6.78 (1H, J=7.8 Hz), 7.39 (1H, d, J=7.4 Hz), 7.42-7.56 (2H, m), 7.72 (1H, d, J=7.4 Hz), 7.84 (1H, d, J=7.8 Hz), 7.94 (1H, s), and 10.08 (1H, bt).

### Step 3. 7-(8-N-Phenylaminooctyl)amino-2H-anthra[1,9-cd]pyrazol-6-one (5d)

The compound 5d (18 mg, 21% yield) was prepared from 4d (72 mg, 0.20 mmol) using the same procedure as 5a. HPLC purification of the above compound yielded 5d as an amorphous red solid; mp 170-172° C. HPLC conditions: Zorbax CN, 5 μm, 21×250 mm, eluting with linear gradient 20% to 40% of isopropanol in hexanes over 40 minutes, and flow rate of 10.0 mL/min with a retention time of 23.13 minutes for 5d. ¹H NMR (400 MHz, CDCl₃): δ 1.24 (6H, m), 1.42-1.50 (2H, m), 1.58-1.62(m, 2H), 1.76-1.86 (2H), 3.30 (4H, m), 5.62 (1H, bs), 6.81 (1H, d, J=8.0 Hz), 7.36-7.40 (1H, m), 7.48-7.52 (1H, m), 7.56-7.61 (3H, m), 7.64-7.68 (11-1, m), 7.86-7.89 (2H, d, J=8.2 Hz), 7.92-7.96 (2H, m), 8.19 (1H, s), 10.10 (1H, bt).

### Example 5

### 7-(7-N-Benzoylaminoheptyl)amino-2H-anthra[1,9-cd]pyrazol-6-one (9a)

### Step 1. 7-(7-Aminoheptyl)amino-2H-anthra[1,9-cd]pyrazol-6-one (6a)

To a stirred solution of 2 (222 mg, 0.87 mmol) in DMSO (4 mL) was added 1,7-diaminoheptane (0.68 g, 5.23 mmol) and the reaction mixture stirred at 130° C. for 6 h. The resultant mixture was cooled to room temperature and was partitioned between chloroform and water. The aqueous layer was extracted with chloroform (3×20 mL). The combined organic layer was dried over anhydrous sodium sulfate, filtered and concentrated under vacuum. MPLC purification (CHCl₃:MeOH:NH₄OH 94:5:1) of the residue gave compound 6a (0.20 g, 66% yield) as an amorphous red solid. ¹H NMR (400 MHz, DMSO-d₆): δ 1.30-1.50 (8H, m), 1.60-1.71 (2H, m), 2.45-2.55 (2H, m), 3.20-3.30 (2H, m), 6.82 (1H, d, J=8.4 Hz), 7.36 (1H, d, J=6.8 Hz), 7.50 (1H, t, J=8.0 Hz), 7.62 (1H, t, J=7.6 Hz), 7.77 (1H, d, J=7.2 Hz), 7.85 (1H, d, J=8.0 Hz) and 10.04 (1H, bt).

### Step 2. 2-Benzyl-7-(7-aminoheptyl)amino-2H-anthra[1,9-cd]pyrazol-6-one (7a)

To the activated 3A molecular sieves powder (0.20 g) in anhydrous DMF (3 mL) was added cesium hydroxide monohydrate (43 mg, 0.29 mmol), and then the white suspension was vigorously stirred for 10 min. After compound 6a (0.10 g, 0.29 mmol) was added and followed by additional 30 min stirring, benzyl bromide (49 mg, 0.29 mmol) was added. After 16 h stirring, the molecular sieves and inorganic salts were filtered off. The filtrate was diluted with DCM (100 mL), washed with brine (3×20 mL), dried over Na₂SO₄, filtered, and concentrated in vacuo. MPLC (CHCl₃:MeOH:NH₄OH 94:5:1) purification of the resulting residue gave the compound 7a (50 mg, 41%) as a amorphous red solid. ¹H NMR (400 MHz, CDCl₃): δ 1.25-1.77 (m, 10H), 2.63-2.70 (m, 2H), 3.24-3.29 (m, 2H), 5.65 (s, 2H), 6.73 (d, J=7.2 Hz, 1H), 7.23-7.47 (m, 9H), 7.82 (d, J=6.8 Hz, 1H), 10.05 (s, 1H). ¹³C NMR (100 MHz, CDCl₃): δ 6 187.25, 153.97, 140.42, 138.91, 136.68, 135.14, 133.29, 129.07, 128.65, 128.41, 128.28, 128.07, 127.66, 123.69, 119.69, 114.42, 112.22, 109.70, 54.32, 43.09, 42.31, 33.70, 29.47, 29.26, 27.47 and 27.03.

### Step 3. 2-Benzyl-7-(7-N-benzoylaminoheptyl)amino-2H-anthra[1,9-cd]pyrazol-6-one (8a)

To a 25 mL round bottom flask, compound 7a (24 mg, 0.05 mmol), benzoic acid (8 mg, 0.66 mmol), HOBt (11 mg, 0.08 mmol), BOP (36 mg, 0.08 mmol) and NMM (8 mg, 0.08 mmol) were added. After the mixture was stirred at rt for 20 h, it was quenched with H₂O and extracted with EtOAc (30 mL) and Et₂O (30 mL). After being washed with H₂O (3×15 mL) and brine (2×10 mL), the organic layer was dried over Na₂SO₄ and concentrated under vacuum. MPLC purification (Hexanes:EtOAc 3:1) afforded the compound 8a (10 mg, 34%) as a red solid. ¹H NMR (400 MHz, CDCl₃): δ 1.45-1.81 (m, 10H), 3.31 (m, 2H), 3.47-3.49 (m, 2H), 5.70 (s, 2H), 6.21 (bs, 1H), 6.78 (d, J=7.6 Hz, 1H), 7.27-7.49 (m, 12H), 7.77 (d, J=7.2 Hz, 2H), 7.84 (d, J=6.8 Hz, 1H) and 10.09 (bs, 1H).

### Step 4. 7-(7-N-Benzoylaminoheptyl)amino-2H-anthra[1,9-cd]pyrazol-6-one (9a)

To a stirred solution of compound 8a (10 mg, 0.018 mmol) in THF (3 mL) and DMSO (0.5 mL) was added t-BuOK (10 mg, 0.089 mmol) and air was bubbled through the mixture at 0° C. Upon completion (determined by TLC) the reaction was quenched with H₂O. The mixture was extracted with EtOAc (100 mL), and the organic layer was washed with brine (3×10 mL), dried over Na₂SO₄ and concentrated under vacuum. The residue was purified by MPLC (Hexanes: EtOAc 3:1) to give the compound 9a (6 mg, 72%) as a red solid. Compound 9a was further purified by using HPLC [Waters Xterra MS C-18, 5 μm, 21×250 mm, eluting with linear gradient of 80% solution A (1000 mL of H₂O and 1 mL TFA) to 100% of solution B (100 mL H₂O, 900 mL of CH₃CN, and 1 mL TFA) over 15 min, and flow rate of 10.0 mL/min with a retention time of 13.52 min]. ¹H NMR (400 MHz, DMSO-d₆): δ 1.36-1.55 (m, 8H), 1.67-1.70 (m, 2H), 3.22-3.27 (m, 4H), 6.84 (d, J=8.8 Hz, 1H), 7.35-7.53 (m, 5H), 7.63 (t, J=7.6 Hz, 1H), 7.76-7.86 (m, 4H), 8.44 (bt, 1H), 10.05 (s, 1H) and 13.59 (s, 1H).

### Example 6

### 7-(9-N-Benzoylaminoheptyl)amino-2H-anthra[1,9-cd]pyrazol-6-one (9b)

Compound 9b (12 mg, 5% overall yield) was prepared by the same scheme as compound 9a. Compound 9b was further purified by using reverse phase HPLC [Waters Xterra MS C-18, 5 μm, 21×250 mm, eluting with linear gradient of 80% solution A (1000 mL of H₂O) to 100% of solution B (100 mL H₂O and 900 mL of CH₃CN) over 20 min, and flow rate of 10.0 mL/min with a retention time of 24.80 min]. ¹H NMR (400 MHz, DMSO-d₆): δ 1.20-1.47 (m, 12H), 1.63-1.68 (m, 211), 3.20-3.28 (m, 4H), 6.83 (d, J=8.8 Hz, 1H), 7.35-7.53 (m, 5H), 7.62 (t, J=7.6 Hz, 1H), 7.77-7.86 (m, 4H), 8.42 (bt, 1H), 10.04 (bt, 1H) and 13.57 (bs, 1H). ¹³C NMR (100 MHz, DMSO-d₆): δ 187.10, 166.70, 153.95, 140.04, 139.47, 125.94, 135.40, 133.68, 131.65, 129.17, 128.90, 127.79, 127.23, 122.22, 119.79, 116.59, 114.11, 112.58, 109.39, 42.73, 39.85, 29.79, 29.65, 29.44, 29.19, 27.32, 27.17.

### Example 7

### 7-(5-(p-Tolyloxy)pentyl)amino-2H-anthra[1,9-cd]pyrazol-6-one (11)

### Step 1. 7-(5-Hydroxy pentyl)amino-2H-anthra[1,9-cd]pyrazol-6-one (10)

To a stirred solution of 2 (0.254 g, 1.00 mmol) in pyridine (5 mL) was added 1-amino-5-pentanol (0.515 g, 5.00 mmol) and the reaction mixture was refluxed for 16 h. The resultant mixture was cooled to room temperature and was partitioned between chloroform and water. The aqueous layer was extracted with chloroform (3×20 mL). The combined organic layer was dried over anhydrous sodium sulfate, filtered and concentrated under vacuum. MPLC purification (CHCl₃:MeOH/99:5) of the residue gave 10 (0.150 g, 47% yield) as an amorphous red solid. ¹H NMR (400 MHz, CD₃OD): δ 1.56-1.64 (4H, m), 1.76-1.82 (2H, m), 3.32 (2H, m), 3.60 (2H, t, J=7.2 Hz), 6.78 (1H, d, J=8.0 Hz), 7.38-7.48 (2H, m), 7.58-7.60(1H, m), 7.70 (1H, m), 7.80 (1H, d, J=8.0 Hz) and 10.0 (1H, bt).

### Step 2. 7-(5-(p-Tolyloxy)pentyl)amino-2H-anthra[1,9-cd]pyrazol-6-one (11)

A solution of 10 (0.045 g, 0.14 mmol), p-cresol (0.030 g, 0.28 mmol) and triphenylphosphine (0.073 g, 0.28 mmol) in anhydrous THF (2 mL) was sonicated for 15 min followed by the addition of DIAD (56 μL, 0.28 mmol). The resulting mixture was further sonicated for another 15 min. The reaction mixture was concentrated and the residue was subjected to MPLC purification (CHCl₃: MeOH/99:1) to give 11 as a red amorphous solid (0.024 mg, 42%); mp 122-125° C.; Compound 11 was further purified by using HPLC [Waters Xterra MS C-18, 5 μm, 21×250 mm, eluting with linear gradient of 80% solution A (1000 mL of H₂O and 1 mL TFA) to 100% of solution B (100 mL H₂O, 900 mL of CH₃CN, and 1 mL TFA) over 20 min, and flow rate of 10.0 mL/min with a retention time of 23.23 min]. ¹H NMR (400 MHz, DMSO-d₆): δ 1.52-1.60 (2H, m), 1.70-1.80 (4H, m), 2.18 (3H,s), 3.30 (2H, t, J=7.4 Hz), 3.92 (2H, t, J=7.4 Hz), 6.78 (2H, d, J=7.8 Hz), 6.82 (1H, d, J=8.0 Hz), 7.02 (2H, d, J=7.8 Hz), 7.36 (1H, d, J=7.8 Hz), 7.50 (1H, t, J=8.0 Hz), 7.64 (1H, t, J=8.0 Hz), 7.78 (1H, d, J=7.4 Hz), 7.84 (1H, d, J=7.6 Hz), 10.06 (1H, bs) and 13.60 (1H, bs).

### Example 8

### Step 1. Construction of a c-Jun Bacterial Expression Vector

The c-Jun bacterial expression vector was constructed using pET-46. Amino acids spanning 1-201 of c-Jun were amplified by PCR and introduced into the pET-46 vector (pHis-c-jun), resulting in a His fusion protein comprising the 5′end of c-Jun.

### Step 2. Purification of the His-c-Jun (1-201) Protein

The pHis-c-Jun was introduced into BL21 E. coli and single colonies were inoculated in 5 ml of LB medium containing ampicillin (LB-amp, 50 mg/mL) as a seed culture. The seed culture (0.5 mL) was inoculated into 50 mL of LB-amp and then cultured until OD600=0.8-1.0. The cells were induced with 0.5 mM of IPTG and culture continued for 4 hr at 25° C. with shaking. Cells were harvested by centrifugation, washed with 1× PBS, lyzed by treatment of 100 μg/mL of lysozyme for 30 min on ice and then run through a French press twice. The cell lysate was recovered by centrifugation at 16,000 rpm for 25 mM at 4° C.; then 200 μL of Ni-agarose was added for a 50% slurry and then a binding assay was performed at room temperature for 1 h. The beads were washed with ice-cold 1× PBS three times and His-c-Jun proteins were eluted with elution buffer (50 mM NaH₂PO₄, 300 mM NaCl, 200 mM imidazole pH 8.0). The eluted His-c-Jun protein was subjected to dialysis at 4° C. overnight in dialysis buffer, aliquoted and then stored at −70° C. until needed.

### Step 3. SP600125 and Active JNK1, JNK2 or JNK3

The JNK inhibitor, SP600125, was purchased from Calbiochem-Novabiochem Corp. The active JNK1, JNK2 and JNK3 proteins were purchased from Millipore (Catalog numbers for JNK1: 14-327, JNK2: 14-329, JNK3: 14-501).

### Step 4. In Vitro Kinase Assay

To deterimine the optimal dose of the JNKs inhibitor, SP600125, for inhibition of JNKs activity, the His-c-Jun (1-201) protein (2 micrograms) was used for an in vitro kinase assay with active JNK1, JNK2, or JNK3 (each 20 ng; Upstate Biotechnology, Inc). Reactions were carried out at 30° C. for 30 min in a mixture containing 50 micromolar unlabeled ATP, 10 μCi [gamma-³²P] ATP and different doses of SP600125. Reactions were stopped by adding 6× SDS sample buffer. For the inhibitory effect of the new potential inhibitors on JNK1 or JNK3 activity, we used 50 micromolar unlabeled ATP and 10 μCi [gamma-³²P] ATP and 10 μM of each compound. SP600125 at 10 μM was used to compare the inhibitory effect. Reactions were carried out at 30° C. for 30 min and samples were boiled and then resolved by 12% SDS-PAGE and visualized by autoradiography.

### Example 9

### 7-(6-Formamidohexyl)amino-2-phenylanthra[1,9-cd]pyrazol-6-one (14b)

### Step 1. 7-Chloro-2-phenylanthra[1,9-cd]pyrazol-6-one (12)

A 7 mL vial was charged with 7-chloro-2H-anthra[1,9-cd]pyrazol-6-one (2, prepared as in Example 1, Step 1; 100 mg, 0.39 mmol), anhydrous K₃PO₄ (181 mg, 0.85 mmol), CuI (4 mg, 0.02 mmol), and 2 mL anhydrous DMF under nitrogen. To this, under nitrogen, was added iodobenzene (66 μL, 0.59 mmol) and N,N′-dimethylethylenediamine (3.6 μL, 0.03 mmol). The vial was tightly capped and then heated at 110° C. for 19 hours. Cooled to room temperature, filtered (Celite), the filtrate was diluted with water (5 mL), extracted with EtOAc (3×5 mL), the combined organic layer was washed with brine (3 mL), dried over MgSO₄, purified by MPLC on silica gel (gradient from Hex to EtOAc) to afford 30 mg (23%) of a bright yellow solid. mp 243-244° C.; ¹H NMR (400 MHz, CDCl₃) δ 8.33 (dd, J=6.3, 2.7 Hz, 1H), 8.07 (d, J=7.2 Hz, 1H), 8.00 (d, J=8.2 Hz, 1H), 7.87 (d, J =7.6 Hz, 2H), 7.71 (t, J=7.8 Hz, 1H), 7.62-7.69 (m, 4H), and 7.41 (t, J=7.4 Hz, 1H); ¹³C NMR (100 MHz, CDCl₃) δ 182.69, 140.38, 140.25, 138.09, 137.50, 134.42, 133.41, 133.35, 130.00, 129.78, 129.42, 127.43, 127.27, 124.36, 122.52, 122.03, 121.88, and 116.64; IR (KBr) 3044, 2921, 2848, and 1659 cm⁻¹; LRMS-EI m/z 330 ([M⁺], 100%); HRMS (ESI) m/z calcd for C₂₀H₁₁N₂ONa (M+Na⁺) 353.0458, found 353.0497.

### Step 2. 7-(6-Aminohexyl)amino-2-phenylanthra[1,9-cd]pyrazol-6-one TFA salt (13b)

A 7 mL vial was charged with 12 (14 mg, 4.23×10-5 mol), hexanediamine (25 mg, 2.12×10⁴ mol), and DMSO (regular, 1 mL). The vial was tightly closed, heated at 120˜130° C. for 2.5 hours. Cooled to room temperature, diluted with 1 mL MeOH, purified by HPLC on a reversed-phase column (Phenomenex Gemini, 5μ, C18 Axia Packed, 250×21.2 mm, 10 mL/min, linear gradient from 20% B to 100% B over 20 min, A=H₂O (0.1% TFA), B=H₂O/MeCN 9:1 (0.1% TFA), collected peak at R_{T}=17.31 minutes) to afford 11 mg (50%) of a dark red solid. mp 185-187° C.; ¹H NMR (400 MHz, CD₃OD) δ 8.04 (d, J=8.4 Hz, 1H), 7.91 (m, 3H), 7.70 (m, 1H), 7.67-7.60 (m, 2H), 7.50-7.41 (m, 3H), 6.88 (t, J=4.9 Hz, 1H), 3.36 (t, J=6.7 Hz, 2H), 2.95 (t, J=7.6 Hz, 2H), 1.83-1.79 (m, 2H), 1.76-1.68 (m, 2H), and 1.60-1.51 (m, 4H); ¹³C NMR (100 MHz, CD₃OD) δ 186.56, 153.64, 141.43, 140.43, 137.55, 135.03, 132.28, 129.58, 129.42, 127.67, 126.61, 124.03, 121.28, 120.06, 115.96, 113.78, 112.53, 109.80, 42.17, 39.48, 28.63, 27.38, 26.79, 26.61, and 26.04; ¹⁹F NMR (376 MHz, DMSO-d₆) δ −73.94; IR (KBr) 3428, 3064, 2938, 2856, 1679, and 1585 cm⁻¹; LRMS-EI m/z 411 ([M⁺], 100%); HRMS (ESI) m/z calcd for C₂₆H₂₇N₄O (M+H⁺) 411.2185, found 411.2166.

### Step 3. 7-(6-Formamidohexyl)amino-2-phenylanthra[1,9-cd]pyrazol-6-one (14b)

Into a stirred solution of 13b (22 mg, 4.19×10⁻⁵ mol) in 3 mL anhydrous DMF was added conc. H₂SO₄ (4.5 μL, 8.38×10⁻⁵ mol), and heated at 120° C. for 6 hours using a 7 mL vial closed with a cap. Cooled to room temperature, diluted with water (5 mL), extracted with EtOAc (3×3 mL), the combined organic layer was washed with brine, dried over MgSO₄, purified by MPLC on silica gel (gradient from Hex to EtOAc) to afford 15.3 mg (83%) of a dark red solid. mp 179-180° C.; ¹H NMR (400 MHz, CDCl₃) δ 10.07 (br t, 1H), 8.18 (s, 1H), 7.94-7.85 (m, 4H), 7.64 (t, J=7.7 Hz, 1H), 7.59-7.55 (m, 3H), 7.48 (t, J=8.0 Hz, 1H), 7.37 (t, J=7.4 Hz, 1H), 6.79 (d, J=8.6 Hz, 1H), 3.35-3.30 (m, 4H), 1.83-1.76 (m, 2H), 1.615-1.51 (m, 4H), and 1.48-1.42 (m, 2H); ¹³C NMR (100 MHz, CDCl₃) δ 187.10, 161.44, 153.93, 142.02, 140.67, 138.09, 137.85, 135.32, 132.84, 129.89, 129.45, 128.28, 126.83, 124.70, 121.73, 120.46, 115.84, 114.41, 112.74, 110.25, 42.91, 38.28, 29.60, 29.03, 27.07, and 26.79; IR (KBr) 3289, 2938, 2856, and 1643 cm⁻¹; LRMS-EI m/z 439 ([M⁺], 100%); HRMS (ESI) m/z calcd for C₂₇H₂₇N₄O₂ (M+H⁺) 439.2134, found 439.2120.

### Example 10

### 7-(5-Formamidopentyl)amino-2-phenylanthra[1,9-cd]pyrazol-6-one (14a)

The compound 14a was prepared by a procedure similar to that described in Example 9. ¹H NMR (400 MHz, CDCl₃): δ 1.50-1.62 (4H, m), 1.76-1.84 (2H, m), 3.26-3.32 (4H, m), 5.78 (1H, bs), 6.78 (1H, d, J=8.0 Hz), 7.38 (1H, m), 7.54-7.60 (3H, m), 7.66-7.74 (2H, m), 7.86 (2H, d, J=7.8 Hz), 7.90-7.96 (2H, m), 8.21 (1H, s) and 10.08 (1H, bt); HRMS (ESI) m/z calcd for C₂₆H₂₅N₄O₂ (M+H⁺) 425.1978, found 425.1961.

### Example 11

### 7-(7-Formamidoheptyl)amino-2-phenylanthra[1,9-cd]pyrazol-6-one (14c)

The compound 14c was prepared by a procedure similar to that described in Example 9. ¹H NMR (400 MHz, CDCl₃): δ 1.30-1.37 (4H, m), 1.44-1.50 (4H, m), 1.68-1.74 (2H, m), 3.14-3.26 (4H, m), 5.70 (bs, 1H), 6.70 (1H, d, J=8.4 Hz), 7.30 (1H, m), 7.40 (1H, m), 7.48-7.58 (4H, m), 7.76-7.86 (4H, m), 8.21 (1H, s) and 9.89 (1H, bt) HRMS (ESI) m/z calcd for C₂₈H₂₉N₄O₂ (M+H⁺) 453.2291, found 453.2281.

### Example 12

### 7-(8-Formamidooctyl)amino-2-phenylanthra[1,9-cd]pyrazol-6-one (14d)

The compound 14d was prepared by a procedure similar to that described in Example 9. ¹H NMR (400 MHz, CDCl₃): δ 1.24 (6H, m), 1.42-1.50 (2H, m), 1.58-1.62(m, 2H), 1.76-1.86 (2H), 3.30 (4H, m), 5.62 (1H, bs), 6.81 (1H, d, J=8.0 Hz), 7.36-7.40 (1H, m), 7.48-7.52 (1H, m), 7.56-7.61 (3H, m), 7.64-7.68 (1H, m), 7.86-7.89 (2H, d, J=8.2 Hz), 7.92-7.96 (2H, m), 8.19 (1H, s), 10.10 (1H, bt); HRMS (ESI) m/z calcd for C₂₉H₃₁N₄O₂ (M+H⁺) 467.2447, found 467.2437.

### Example 13

### Alternative Synthesis of 7-(6-N-Phenylaminohexyl)amino-2H-anthra[1,9-cd]pyrazol-6-one (5b) using Scheme 5

### 2-(6-(Phenylamino)hexyl)isoindoline-1,3-dione (15)

To a stirred solution of aniline (364 mg, 4 mmol) in CH₃CN (20 mL), was added 2-(6-bromohexyl)isoindoline-1,3-dione (1244 mg, 4 mmol) and K₂CO₃ (552 mg, 4 mmol). The mixture was heated under reflux for 25 hours. The insolubles were filtered off (Celite), the filtrate was concentrated in vacuo. The residue was purified by MPLC to give 15 (500 mg, 39%) as a colorless oil. ¹H NMR (400 MHz, CDCl₃): 8 7.83-7.81 (m, 2H), 7.71-7.68 (m, 2H), 7.15 (dd, J=7.4, 8.4 Hz, 2H), 6.66 (t, J=7.4 Hz, 1H), 6.59 (d, J=8.4 Hz, 2H), 3.68 (t, J=7.2 Hz, 2H), 3.60 (brs, 1H), 3.08 (t, J=7.2 Hz, 2H), 1.73-1.66 (m, 2H), 1.62-1.57 (m, 2H), and 1.47-1.36 (m, 4H); ¹³C NMR (100 MHz, CDCl₃) δ 168.70, 148.71, 134.14, 132.36, 129.44, 123.41, 117.26, 112.90, 43.98, 38.07, 29.58, 28.77, 26.88, and 26.84; LRMS-EI m/z (%) 322 (M⁺, 43%), and 106 (100%).

### 1-N-Phenylhexane-1,6-diamine (16)

To a stirred solution of 15 (500 mg, 1.55 mmol) in EtOH (15 mL) was added hydrazine monohydrate (0.62 mL). After 15 hours of stirring at room temperature and the white insolubles were filtered off (Celite). The filtrate was concentrated in vacuo, the residue was diluted with CHCl₃ (10 mL) and filtered again. Concentration of the filtrate gave 16 (270 mg, 91%) as a colorless oil. ¹H NMR (400 MHz, CDCl₃) δ 7.16 (dd, J=7.4, 8.6 Hz, 2H), 6.67 (t, J=7.4 Hz, 1H), 6.58 (d, J=8.6 Hz, 2H), 3.08 (t, J=8.0 Hz, 2H), 2.64 (t, J=6.8 Hz, 2H), 1.62-1.56 (m, 2H), and 1.47-1.30 (m, 6H); ¹³C NMR (100 MHz, CDCl₃) δ 148.73, 129.45, 117.28, 112.90, 44.11, 42.30, 33.85, 29.75, 27.27, and 26.95; LRMS-EI m/z (%) 192 (M⁺, 32%), and 106 (100%).

### 7-(6-N-Phenylaminohexyl)amino-2H-anthra[1,9-cd]pyrazol-6-one (5b)

To a stirred solution of 2 (prepared as in Example 1, Step 1; 71 mg, 0.28 mmol) in dry pyridine (4 mL) was added 16 (270 mg, 1.40 mmol) and the reaction mixture was stirred at reflux for 24 hours. The solvent was removed in vacuo. MPLC purification (CHCl₃:MeOH:NH₄OH 94:5:1) of the residue gave compound 5b (7 mg, 6% yield) as a red solid foam. Compound 5b was further purified by HPLC (eluting time: 20 minutes for analytical and 40 minutes for semi-preparative column). The semi-preparative and analytical HPLC retention times of 9 were 23.67 and 14.87 minutes, respectively. ¹H NMR (400 MHz, DMSO-d₆) δ 13.59 (brs, 1H), 10.04 (brs, 1H), 7.85 (d, J=8.0 Hz, 1H), 7.76 (d, J=7.6 Hz, 1H), 7.63 (dd, J=7.6, 8.0 Hz, 1H), 7.51 (t, J=7.6, 8.4 Hz, 1H), 7.36 (d, J=7.6 Hz, 1H), 7.26 (t, J=7.6 Hz, 2H), 7.02-6.90 (m, 3H), 6.83 (d, J=8.4 Hz, 1H), 3.28 (t, J=6.6 Hz, 2H), 3.13 (t, J=6.8 Hz, 2H), 1.73-1.64 (m, 2H), 1.63-1.60 (m, 2H), and 1.52-1.36 (m, 4H); ¹³C NMR (100 MHz, CDCl₃) δ 186.74, 153.59, 140.60, 138.35, 136.44, 135.18, 131.59, 130.45, 130.18, 129.39, 127.91, 122.77, 122.11, 120.53, 115.27, 114.22, 113.43, 110.49, 53.01, 42.87, 28.72, 26.78, 26.30, and 25.94; IR (cm⁻¹) 3415.7, 3240.0, 2925.3, 2851.8, 1679.0, 1601.4, 1200.9, 1176.4, 1139.6; EI-MS m/z (%): 411 (M+H⁺, 20%), 318 (37%), and 176 (100%); HRMS-ESI calculated for C₂₆H₂₇N₄O⁺ [M+H⁺] 411.2107, found 411.2163.

A number of embodiments of the invention have been described. Nevertheless, it will be understood that various modifications may be made without departing from the spirit and scope of the invention. Accordingly, other embodiments are within the scope of the following claims. It is to be further appreciated that certain features of the disclosure, which are, for clarity, described in the context of separate embodiments, can also be provided in combination in a single embodiment. Conversely, various features which are, for brevity, described in the context of a single embodiment, can also be provided separately or in any suitable sub-combination.

What is claimed is:

1. A compound of Formula I: 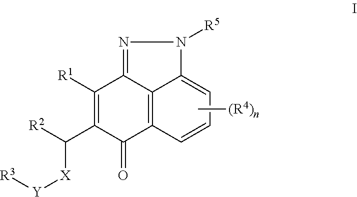
or a pharmaceutically acceptable salt thereof, wherein:
R¹ and R⁴ are independently selected from H, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}; or
R¹ and R⁴ are independently selected from C₁₋₁₀ alkyl, C₂₋₆ alkenyl, C₂₋₆ alkynyl, C₆₋₁₂ aryl, C₅₋₁₂ heteroaryl, C₃₋₁₂ cycloalkyl, C₃.₁₀ heterocycloalkyl, heterocycloalkylalkyl, arylalkyl, and heteroarylalkyl, each optionally substituted with 1, 2, 3, 4, or 5 substituents selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1};
R² is selected from H, C(O)R^{b1}, C(O)NR^{c1}R^{d1}, and C(O)OR^{a1}; or
R² is selected from C₁₋₁₀ alkyl, C₂₋₆ alkenyl, C₂₋₆ alkynyl, C₆₋₁₂ aryl, C₅₋₁₂ heteroaryl, C₃₋₁₂ cycloalkyl, C₃₋₁₀ heterocycloalkyl, heterocycloalkylalkyl, arylalkyl, and heteroarylalkyl, each optionally substituted with 1, 2, 3, 4, or 5 substituents selected from halo, CN, NO₂, OR^{a1}, SR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}; or,
R¹ and R² together with the three C atoms between them may form a 5 or 6 membered cycloalkyl, aryl, or heteroaryl ring each optionally substituted with 1, 2, or 3 substituents independently selected from C₁₋₆ alkyl, C₂₋₆ alkenyl, C₂₋₆ alkynyl, C₁₋₆ alkoxy, C₁₋₆ haloalkyl, halo, OH, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1};
R³ is selected from OR^{a2}, SR^{a2}, NR^{c2}R^{d2}, NR^{c2}C(O)R^{b2}; NR^{c2}C(O)NR^{c2}R^{d2}, NR^{c2}C(O)OR^{a2}, NR^{c2}S(O)₂R^{b2}, C(O)C₁₋₆ alkyl, C(O)C₆₋₁₂ aryl, C(O)NR^{c2}R^{d2}, C(O)OR^{a2}, OC(O)R^{b2}, OC(O)NR^{c2}R^{d2}, S(O)R^{b2}, S(O)NR^{c2}R^{d2}, S(O)₂R^{b2}, and S(O)₂NR^{c2}R^{d2};
X═O, S, C(O), or NR⁶;
Y is a divalent moiety selected from C₃₋₁₂ alkylene, C₂₋₁₀ alkenylene, C₂₋₈ alkynylene, C₃₋₁₀ cycloalkylene, C₃₋₁₀ heterocycloalkylene, C₆₋₁₀ arylene, and C₅₋₁₀ heteroarylene, each optionally substituted by 1, 2 or 3 substituents independently selected from C₁₋₄ alkyl, C₁₋₄ hydroxyalkyl, C₁₋₄ cyano alkyl, C₁₋₄ haloalkyl, C₁₋₄ alkoxy-C₁₋₄ alkyl, C₂₋₄ alkenyl, C₂₋₄ alkynyl, C₃₋₁₀ cycloalkyl, C₃₋₁₀ heterocycloalkyl, C₆₋₁₀ aryl, C₅₋₁₀ heteroaryl, halo, CN, NO₂, SCN, OH, C₁₋₄alkoxy, C₁₋₄ haloalkoxy, amino, C₁₋₄ alkylamino, and C₂₋₈ dialkylamino;
R⁵ and R⁶ are independently selected from H, C₁₋₆ alkyl, C₁₋₄ alkoxy-C₁₋₄ alkyl, C(O)C₁₋₆ alkyl, aryl, heteroaryl, C₇₋₁₈ arylalkyl, and C(O)C₆₋₁₂ aryl;
R^{a1} and R^{a2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, CN, amino, halo, C₁₋₆ alkyl, C₁₋₆ alkoxy, C₁₋₆ haloalkyl, and C ₁₋₆ haloalkoxy;
R^{b1} and R^{b2} are independently selected from H, C₁₋₆ alkyl, C₁₋₆ haloalkyl, C₂₋₆ alkenyl, C₂₋₆ alkynyl, aryl, cycloalkyl, heteroaryl, and heterocycloalkyl, wherein said C₁₋₆ alkyl, C₂₋₆ alkenyl, C₂₋₆ alkynyl, aryl, cycloalkyl, heteroaryl, or heterocycloalkyl is optionally substituted with 1, 2, or 3 substituents independently selected from OH, C₁₋₆ alkoxy, CN, amino, alkylamino, dialkylamino, halo, C₁₋₆ alkyl, C₁₋₆ haloalkyl, aryl, arylalkyl, heteroaryl, heteroarylalkyl, cycloalkyl, and heterocycloalkyl;
R^{c1}, R^{c2}, R^{d1}, and a R^{d2} are independently selected from H, C₁₋₆ alkyl, C₂₋₆ alkenyl, C₂₋₆ alkynyl, aryl, cycloalkyl, heteroaryl, and heterocycloalkyl, wherein said C₁₋₆ alkyl, C₂₋₆ alkenyl, C₂₋₆ alkynyl, aryl, cycloalkyl, heteroaryl, or heterocycloalkyl is optionally substituted with 1, 2, or 3 substituents independently selected from OH, C₁₋₆ alkoxy, CN, amino, alkylamino, dialkylamino, halo, C₁₋₆ alkyl, C₁₋₆ haloalkyl, aryl, arylalkyl, heteroaryl, heteroarylalkyl, cycloalkyl, and heterocycloalkyl; or,
R^{c1} and R^{d1}, or R^{c2} and R^{d2}, together with the N atom to which they are attached, may optionally form a 4-, 5-, 6-, or 7-membered heterocycloalkyl group or heteroaryl group, each optionally substituted with 1, 2, or 3 substituents independently selected from OH, C₁₋₆ alkoxy, CN, amino, alkylamino, dialkylamino, halo, C₁₋₆ alkyl, C₁₋₆ haloalkyl, aryl, arylalkyl, heteroaryl, heteroarylalkyl, cycloalkyl, and heterocycloalkyl; and
n is 1, 2, or 3.

2. The compound of claim 1, or a pharmaceutically acceptable salt thereof, wherein R¹ and R⁴ are independently selected from H, halo, CN, NO₂, OR^{a1}, and SR^{a1}.

3. The compound of claim 1, or a pharmaceutically acceptable salt thereof, wherein R¹ and R⁴ are independently selected from H, C(O)R^{b1}, C(O)NR^{c1}R^{d1}, C(O)OR^{a1}, OC(O)R^{b1}, and OC(O)NR^{c1}R^{d1}.

4. The compound of claim 1, or a pharmaceutically acceptable salt thereof, wherein R¹ and R⁴ are independently selected from H, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}.

5. The compound of claim 1, or a pharmaceutically acceptable salt thereof, wherein R¹ and R⁴ are independently selected from C₁₋₁₀ alkyl, C₂₋₆ alkenyl, C₂₋₆ alkynyl, C₆₋₁₂ aryl, C₃₋₁₂ cycloalkyl, and arylalkyl, each optionally substituted with 1, 2, 3, 4, or 5 substituents selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}.

6. The compound of claim 1, or a pharmaceutically acceptable salt thereof, wherein R¹ and R⁴ are independently selected from C₅₋₁₂ heteroaryl, C₃₋₁₀ heterocycloalkyl, heterocycloalkylalkyl, and heteroarylalkyl, each optionally substituted with 1, 2, 3, 4, or 5 substituents selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1},

7. The compound of claim 1, or a pharmaceutically acceptable salt thereof, wherein R² is selected from H, C(O)R^{b1}, C(O)NR^{c1}R^{d1}, and C(O)OR^{a1}.

8. The compound of claim 1, or a pharmaceutically acceptable salt thereof, wherein R² is selected from C₁₋₁₀ alkyl, C₂₋₆ alkenyl, C₂₋₆ alkynyl, C₆₋₁₂ aryl, C₃₋₁₂ cycloalkyl, and arylalkyl, each optionally substituted with 1, 2, 3, 4, or 5 substituents selected from halo, CN, NO₂, OR^{a1}, SR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}.

9. The compound of claim 1, or a pharmaceutically acceptable salt thereof, wherein R² is selected from C₅₋₁₂heteroaryl, C₃₋₁₀heterocycloalkyl, heterocycloalkylalkyl, and heteroarylalkyl, each optionally substituted with 1, 2, 3, 4, or 5 substituents selected from halo, CN, NO₂, OR^{a1}, SR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}.

10. The compound of claim 1, or a pharmaceutically acceptable salt thereof, wherein R¹ and R² together with the three C atoms between them may form a 5, 6, or 7 membered cycloalkyl ring optionally substituted with 1, 2, or 3 substituents independently selected from C₁₋₆ alkyl, C₂₋₆ alkenyl, C₂₋₆ alkynyl, C₁₋₆ alkoxy, C₁₋₆ haloalkyl, halo, OH, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}.

11. The compound of claim 1, or a pharmaceutically acceptable salt thereof, wherein R¹ and R² together with the three C atoms between them may form a 6 membered aryl ring optionally substituted with 1, 2, or 3 substituents independently selected from C₁₋₆ alkyl, C₂₋₆ alkenyl, C₂₋₆ alkynyl, C₁₋₆ alkoxy, C₁₋₆ haloalkyl, halo, OH, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}.

12. The compound of claim 1, or a pharmaceutically acceptable salt thereof, wherein R¹ and R² together with the three C atoms between them may form a 5 or 6 membered heteroaryl ring optionally substituted with 1, 2, or 3 substituents independently selected from C₁₋₆ alkyl, C₂₋₆ alkenyl, C₂₋₆ alkynyl, C₁₋₆ alkoxy, C₁₋₆ haloalkyl, halo, OH, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}.

13. The compound of claim 1, or a pharmaceutically acceptable salt thereof, wherein R¹ and R² together with the three C atoms between them may form a 6-membered aryl ring optionally substituted with 1, 2, or 3 substituents independently selected from C₁₋₆ alkyl, C₂₋₆ alkenyl, C₂₋₆ alkynyl, C₁₋₆ alkoxy, C₁₋₆ haloalkyl, halo, OH, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1},NR^{c1}C(O)OR^{a1}, NR^{c1}C(O)NR^{c1}R^{d1}, NR^{c1}S(O)₂R^{b1}, S(O)R^{b1}, S(O)₂R^{b1}, and S(O)₂NR^{c1}R^{d1}.

14. The compound of claim 1, or a pharmaceutically acceptable salt thereof, wherein R³ is selected from OR^{a2}, SR^{a2}, NR^{c2}R^{d2}, NR^{c2}C(O)R^{b2}, NR^{c2}C(O)NR^{c2}R^{d2}, NR^{c2}C(O)OR^{a2}, and NR^{c2}S(O)₂R^{b2}.

15. The compound of claim 1, or a pharmaceutically acceptable salt thereof, wherein R³ is selected from OR^{a2}, NR^{c2}R^{d2}, NR^{c2}C(O)R^{b2}, NR^{c2}C(O)OR^{a2}, C(O)C₁₋₆ alkyl, C(O)C₆₋₁₂ aryl, C(O)NR^{c2}R^{d2}, C(O)OR^{a2}, OC(O)R^{b2}, OC(O)NR^{c2}R^{d2}, S(O)R^{b2}, S(O)NR^{c2}R^{d2}, S(O)₂R^{b2}, and S(O)₂NR^{c2}R^{d2}.

16. The compound of claim 1, or a pharmaceutically acceptable salt thereof, wherein R³ is selected from OR^{a2}, NR^{c2}R^{d2}, NR^{c2}_{C(O)OR}^{b2}, and NR^{c2}C(O)OR^{a2}.

17. The compound of claim 1, or a pharmaceutically acceptable salt thereof, wherein R³ is NH—CHO.

18. The compound of claim 1, or a pharmaceutically acceptable salt thereof, wherein X═O, S, C(O), or NR⁶.

19. The compound of claim 1, or a pharmaceutically acceptable salt thereof, wherein X═O.

20. The compound of claim 1, or a pharmaceutically acceptable salt thereof, wherein X═S.

21. The compound of claim 1, or a pharmaceutically acceptable salt thereof, wherein X═NR⁶.

22. The compound of claim 1, or a pharmaceutically acceptable salt thereof, wherein Y is a divalent moiety selected from C₃₋₁₂ alkylene, C₂₋₁₀ alkenylene, C₂₋₈ alkynylene, C₃₋₁₀ cycloalkylene, and C₆₋₁₀ arylene, each optionally substituted by 1, 2 or 3 substituents independently selected from C₁₋₄ alkyl, C₁₋₄ hydroxyalkyl, C₁₋₄ cyanoalkyl, C₁₋₄ haloalkyl, C₁₋₄ alkoxy-C₁₋₄ alkyl, C₂₋₄ alkenyl, C₂₋₄ alkynyl, C₃₋₁₀ cycloalkyl, C₃₋₁₀ heterocycloalkyl, C₆₋₁₀ aryl, C₅₋₁₀ heteroaryl, halo, CN, NO₂, SCN, OH, C₁₋₄alkoxy, C₁₋₄ haloalkoxy, amino, C₁₋₄ alkylamino, and C₂₋₈ dialkylamino.

23. The compound of claim 1, or a pharmaceutically acceptable salt thereof, wherein Y is a divalent moiety selected from C₃₋₁₂ alkylene, C₃₋₁₀ heterocycloalkylene, and C₅₋₁₀ heteroarylene, each optionally substituted by 1, 2 or 3 substituents independently selected from C₁₋₄ alkyl, C₁₋₄ hydroxyalkyl, C₁₋₄ cyanoalkyl, C₁₋₄ haloalkyl, C₁₋₄ alkoxy-C₁₋₄ alkyl, C₂₋₄ alkenyl, C₂₋₄ alkynyl, C₃₋₁₀ cycloalkyl, C₃₋₁₀ heterocycloalkyl, C₆₋₁₀ aryl, C₅₋₁₀ heteroaryl, halo, CN, NO₂, SCN, OH, C₁₋₄alkoxy, C₁₋₄ haloalkoxy, amino, C₁₋₄ alkylamino, and C₂₋₈ dialkylamino.

24. The compound of claim 1, or a pharmaceutically acceptable salt thereof, wherein Y is C₃₋₁₂ alkylene optionally substituted by 1, 2 or 3 substituents independently selected from C₁₋₄ alkyl, C₁₋₄ hydroxyalkyl, C₁₋₄ cyanoalkyl, C₁₋₄ haloalkyl, C₁₋₄ alkoxy-C₁₋₄ alkyl, C₂₋₄ alkenyl, C₂₋₄ alkynyl, C₃₋₁₀ cycloalkyl, C₃₋₁₀ heterocycloalkyl, C₆₋₁₀ aryl, C₅₋₁₀ heteroaryl, halo, CN, NO₂, SCN, OH, C₁₋₄alkoxy, C₁₋₄ haloalkoxy, amino, C₁₋₄ alkylamino, and C₂₋₈ dialkylamino.

25. The compound of claim 1, or a pharmaceutically acceptable salt thereof, wherein R⁵ and R⁶ are independently selected from H, C₁₋₄ alkoxy-C₁₋₄ alkyl, C(O)C₁₋₆ alkyl, C₇₋₁₈ arylalkyl, and C(O)C₆₋₁₂ aryl.

26. The compound of claim 1, or a pharmaceutically acceptable salt thereof, wherein R⁵ and R⁶ are independently selected from H, C₁₋₆ alkyl, aryl, and heteroaryl.

27. The compound of claim 1, or a pharmaceutically acceptable salt thereof, wherein R⁵ and R⁶ are independently H or C₇₋₁₈ arylalkyl.

28. The compound of claim 1, or a pharmaceutically acceptable salt thereof, wherein R⁵ is aryl.

29. The compound of claim 1, or a pharmaceutically acceptable salt thereof, wherein n is 1.

30. The compound of claim 1, having the Formula: 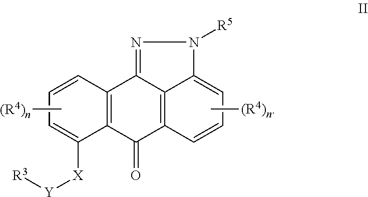
or a pharmaceutically acceptable salt thereof.

31. The compound of claim 1, having the Formula: 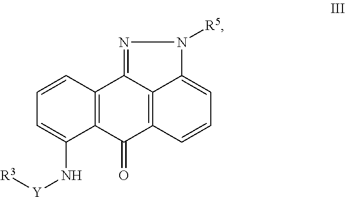
or a pharmaceutically acceptable salt thereof.

32. The compound of claim 1, having the Formula VI: 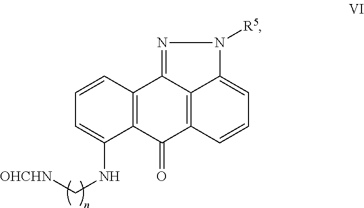
or a pharmaceutically acceptable salt thereof.

33. The compound of claim 1, selected from:
7-(5-Aminopentyl)amino-2H-anthra[1,9-cd]pyrazol-6-one;
7-(6-N-Boc-Aminohexyl)amino-2H-anthra[1,9-cd]pyrazol-6-one;
7-(7-N-Boc-Aminoheptyl)amino-2H-anthra[1,9-cd]pyrazol-6-one;
7-(8-N-Boc-Aminooctyl)amino-2H-anthra[1,9-cd]pyrazol-6-one;
7-(6-Aminohexyl)amino-2H-anthra[1,9-cd]pyrazol-6-one;
7-(7-Aminoheptyl)amino-2H-anthra[1,9-cd]pyrazol-6-one;
7-(8-Aminooctyl)amino-2H-anthra[1,9-cd]pyrazol-6-one;
2-Benzyl-7-(7-aminoheptyl)amino-2H-anthra[1,9-cd]pyrazol-6-one;
2-Benzyl-7-(7-N-benzoylaminoheptyl)amino-2H-anthra[1,9-cd]pyrazol-6-one;
7-(7-N-Benzoylaminoheptyl)amino-2H-anthra[1,9-cd]pyrazol-6-one;
7-(9-N-Benzoylaminoheptyl)amino-2H-anthra[1,9-cd]pyrazol-6-one;
7-(5-Hydroxy pentyl)amino-2H-anthra[1,9-cd]pyrazol-6-one;
7-(5-(p-Tolyloxy)pentyl)amino-2H-anthra[1,9-cd]pyrazol-6-one;
7-(5-Formamidopentyl)amino-2-phenylanthra[1,9-cd]pyrazol-6-one;
7-(6-Formamidohexyl)amino-2-phenylanthra[1,9-cd]pyrazol-6-one;
7-(7-Formamidoheptyl)amino-2-phenylanthra[1,9-cd]pyrazol-6-one; and
7-(8-Formamidooctyl)amino-2-phenylanthra[1,9-cd]pyrazol-6-one, or a pharmaceutically acceptable salt thereof.

34. A composition comprising a compound according to claim 1, or a pharmaceutically acceptable salt thereof, and at least one pharmaceutically acceptable carrier.

35. A method of modulating an activity of JNK1, the method comprising contacting JNK1 with a compound of claim 1, or a pharmaceutically acceptable salt thereof.

36. The method of claim 35 wherein said compound is a selective inhibitor of JNK1 over JNK2 and JNK3.

37. The method of claim 35, wherein the compound, or a pharmaceutically acceptable salt thereof, has a Formula IV: 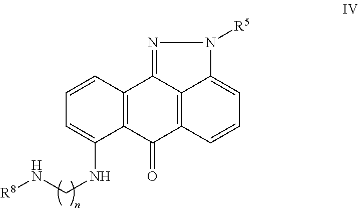
wherein:
(i) R⁸═C₆H₅, n=4-9; or
(ii) R⁸═C₆H₅C(O), n=9.

38. A method of modulating an activity of JNK3, the method comprising contacting JNK3 with a compound of claim 1, or a pharmaceutically acceptable salt thereof.

39. The method of claim 38 wherein said compound, or a pharmaceutically acceptable salt thereof, is a selective inhibitor of JNK3 over JNK1 and JNK2.

40. The method of claim 38, wherein the compound, or a pharmaceutically acceptable salt thereof, has a Formula V: 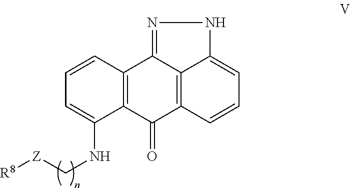
wherein:
(i) R⁸═C₆H₅, Z═NH, n=4-9; or
(ii) R⁸═4-CH₃—C₆H₄, Z═O, n=4-9; or
(iii) R⁸═C₆H₅C(O), Z═NH, n=4-9.

41. A method of modulating an activity of JNK1 and JNK2, the method comprising contacting JNK1 and JNK2 with a compound of claim 1, or a pharmaceutically acceptable salt thereof.

42. The method of claim 41 wherein said compound is a selective inhibitor of JNK1 and JNK2 over JNK3.

43. The method of claim 42, wherein the compound, or a pharmaceutically acceptable salt thereof, has a Formula VI:
wherein, n=3-10.

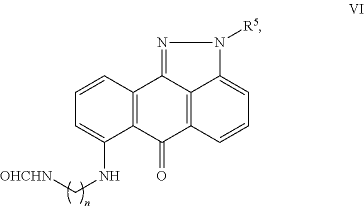

44. The method of claim 42, wherein the compound is: 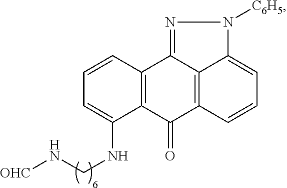
or a pharmaceutically acceptable salt thereof.

45. A method for treating, preventing, or ameliorating one or more symptoms associated with type-2 diabetes, insulin resistance, neural degeneration, or rheumatoid arthritis, the method comprising administering to a subject in need thereof a therapeutically effective amount of a compound of claim 1, or a pharmaceutically acceptable salt thereof.
